(19) United States
(12) Patent Application Publication
(10) Pub. No.: US 2026/0345032 A1
(43) Pub. Date: Nov. 5, 2026

(54) SYSTEMS AND METHODS FOR BIOMECHANICAL ANALYSIS OF MOTION

(71) Applicant: Flyte, Inc., Miami, FL (US)

(72) Inventors: Royi Metser, Miami, FL (US); Rahul Matta, Newton, MA (US); Fabian Hoitz, Calgary (CA); Sam Blades, Victoria (CA); Peter Ruppe, Scottsdale, AZ (US)

(21) Appl. No.: 19/665,812

(22) Filed: May 1, 2026

Related U.S. Application Data
(60) Provisional application No. 63/798,448, filed on May 1, 2025.

(51) Int. Cl. A61B 5/11; A61B 5/00

(52) U.S. Cl. CPC A61B 5/112; A61B 5/002; A61B 5/6807; A61B 5/7275; A61B 2562/0219

(57) ABSTRACT

A gait monitoring system includes first and second monitoring devices respectively associated with first and second feet of a user and positioned within footwear. Each monitoring device includes at least one inertial sensor, a processor, memory, a timing circuit, and a wireless communication architecture, and is configured to acquire inertial data during an activity. The monitoring devices operate independently during the activity and store time-associated data locally. Inertial data is converted to stride data at the monitoring devices and transferred to a computing device and associated based on relative timing using correlation parameters to align first-foot and second-foot data. The system determines step events, phase-based gait metrics including stance and swing phases, and bilateral gait characteristics based on the associated data. Derived metrics are compared to a baseline gait profile and used to determine cumulative impact and deviations from typical gait behavior.

### CROSS-REFERENCE TO RELATED APPLICATION

This application claims priority to U.S. Provisional Patent Application No. 63/798,448, filed May 1, 2025 and entitled “Systems and Methods for Biomechanical Analysis of Running Motion,” which is incorporated by reference herein in its entirety for all purposes.

### BACKGROUND

There are a variety of devices that provide information about exercise, such as smart watches, applications executing on smartphones, and custom devices that attach to clothing of a user or to an exercise device (e.g., a bicycle). Such devices can provide useful information about a user's activity or exercise, such as pulse rate, distance, speed, and the like. However, such devices typically have a variety of deficiencies in the collection and calculation of accurate biomechanical data. Many current wearables are also intrusive, bulky, and/or difficult to use. As a result, the wearer may not use the devices for an extended period of time. The types of data that are collected from such devices, and thus the derived information that can be determined from such data, are limited by sensor types, sensor locations, and the types of calculated and derived metrics that can be derived therefrom, limiting their utility for accurately and reliably determining activity and movement and providing users with effective guidance to improve health and performance. Further, many conventional systems are unable to robustly associate movement data acquired from a first foot with movement data acquired from a second foot, thereby limiting determination of bilateral gait characteristics, temporal symmetry characteristics, phase-based running metrics, individualized training recommendations, and injury-related insights.

### SUMMARY

According to an example embodiment, the system includes a first self-contained monitoring device that is configured to be secured relative to a first foot of a user and to generate first stride data from inertial data acquired by a first inertial measurement unit during an activity. The system includes a second self-contained monitoring device that is configured to be secured relative to a second foot of the user and to generate second stride data from inertial data acquired by a second inertial measurement unit during the activity. The system also includes one or more computing devices that are separate from the first monitoring device and the second monitoring device. The one or more computing devices are configured to receive the first stride data for the activity and the second stride data for the activity, to associate the first stride data with the second stride data based on relative timing between the first stride data and the second stride data during the activity to generate an associated bilateral data set, to determine, from the associated bilateral data set, a mechanical load metric representing cumulative physical stress experienced by the user during the activity, and to provide one or more representations of the mechanical load metric to the user.

In another example embodiment, the system includes a first monitoring device that is configured to be secured relative to a first foot of a user, to generate first stride data records from inertial data acquired by a first inertial measurement unit during an activity, and to store a first local data set comprising the first stride data records in a first local memory of the first monitoring device. The system includes a second monitoring device that is configured to be secured relative to a second foot of the user, to generate second stride data records from inertial data acquired by a second inertial measurement unit during the activity, and to store a second local data set comprising the second stride data records in a second local memory of the second monitoring device. The system also includes one or more computing devices that are configured to receive the first local data set from the first local memory and the second local data set from the second local memory, to associate the first local data set with the second local data set based on relative timing between the first local data set and the second local data set to generate an associated bilateral data set, with the first local data set and the second local data set having been generated and stored independently by the first and second monitoring devices during the activity, to generate a baseline gait profile that comprises expected ranges for one or more gait metrics of the user, to determine, from the associated bilateral data set, a cumulative mechanical load metric for the activity, to compare one or more current gait metrics from the activity to the expected ranges of the baseline gait profile, and to generate a recommendation for the user based at least in part on the cumulative mechanical load metric and the comparison of the current gait metrics to the expected ranges.

In another example embodiment, the system includes a first monitoring device that is configured to be secured relative to a first foot of a user, to acquire first inertial data from a first inertial measurement unit during an activity, and to generate and store in a first local memory first stride data records, with each first stride data record comprising at least timestamps for initial contact, midstance, and toe-off of a respective stride of the first foot and at least pronation excursion and foot strike angle of the respective stride of the first foot determined from raw inertial signals that are not retained in the first local memory after generation of the respective first stride data record during the activity. The system also includes a second monitoring device that is configured to be secured relative to a second foot of the user, to acquire second inertial data from a second inertial measurement unit during the activity, and to generate and store in a second local memory second stride data records, with each second stride data record comprising at least timestamps for initial contact, midstance, and toe-off of a respective stride of the second foot and at least pronation excursion and foot strike angle of the respective stride of the second foot determined from raw inertial signals that are not retained in the second local memory after generation of the respective second stride data record during the activity. The system further includes one or more computing devices that are configured to receive the first stride data records from the first local memory and the second stride data records from the second local memory after the activity, to associate the first stride data records with the second stride data records based on relative timing, including by performing a coarse alignment based on timing information derived from each of the first monitoring device and the second monitoring device and a finer alignment using detected step events or contact events as alignment anchors, to generate an associated bilateral data set, to determine, from the associated bilateral data set, an estimated peak vertical ground reaction force for each of a plurality of locomotion intervals to determine, for each locomotion interval, an impact-related value based on the estimated peak vertical ground reaction force over a stance phase of the respective locomotion interval, to determine a cumulative impact metric for the activity by aggregating the impact-related values, to compare the cumulative impact metric to a threshold derived from historical activity data of the user, to establish a baseline gait profile comprising expected ranges for one or more gait metrics of the user and compare one or more current gait metrics from the activity to the expected ranges, and to generate, based at least in part on the cumulative impact metric comparison and the gait comparison, a recommendation for the user.

### BRIEF DESCRIPTION OF DRAWINGS

The above and other features of the present disclosure, its nature, and various advantages will be more apparent upon consideration of the following detailed description, taken in conjunction with the accompanying drawings in which:

FIG. 1 shows a side plan view of an illustrative footwear assembly in accordance with an embodiment of the present disclosure;

FIG. 2 depicts an exemplary insole and sole from the footwear assembly of FIG. 1 in accordance with an embodiment of the present disclosure;

FIG. 3 depicts an exploded view of an exemplary monitoring device from the footwear assembly of FIG. 1 in accordance with an embodiment of the present disclosure;

FIG. 4 depicts a perspective interior view of the monitoring device from FIG. 3 in accordance with an embodiment of the present disclosure;

FIG. 5 depicts a top plan interior view of the monitoring device from FIG. 3 in accordance with an embodiment of the present disclosure;

FIG. 6 depicts a pairing and connecting system diagram for the footwear assembly of FIG. 1 in accordance with an embodiment of the present disclosure;

FIG. 7 depicts a paired-connection system diagram for the footwear assembly of FIG. 1 in accordance with an embodiment of the present disclosure;

FIG. 8 depicts a synchronization and run-data workflow/system diagram for the footwear assembly of FIG. 1 in accordance with an embodiment of the present disclosure;

FIG. 9 depicts exemplary steps of a process for on-device stride detection and stride record generation in accordance with an embodiment of the present disclosure;

FIG. 10 depicts exemplary steps of a process for on-device stride data determinations at the monitoring device in accordance with an embodiment of the present disclosure;

FIG. 11 depicts exemplary steps of a process for associating first stride data and second stride data and identifying bilateral stride event information in accordance with an embodiment of the present disclosure;

FIG. 12 depicts exemplary steps of a process for phase-based gait metric determination using the associated bilateral data set in accordance with an embodiment of the present disclosure;

FIG. 13 depicts exemplary steps of a process for bilateral symmetry and gait characteristic determination in accordance with an embodiment of the present disclosure;

FIG. 14 depicts exemplary steps of a process for baseline gait profile generation, cumulative impact determination, and recommendation generation in accordance with an embodiment of the present disclosure;

FIG. 15 depicts an example user interface showing impact-related score information in accordance with an embodiment of the present disclosure;

FIG. 16 depicts an example user interface showing resiliency-related or readiness-related information in accordance with an embodiment of the present disclosure; and

FIG. 17 depicts an example user interface showing deviation, anomaly, and recommendation information in accordance with an embodiment of the present disclosure.

### DETAILED DESCRIPTION

The present disclosure relates to a system for monitoring how a person moves while walking or running, for example, using small monitoring devices in footwear. In one example, a first device is associated with the user's first foot and a second device is associated with the user's second foot. Each device can be located below the foot, such as in or near the sole or insole of a shoe or other footwear article. Because the devices are positioned close to the point where the foot interacts with the ground, the system can collect motion information that is closely tied to the user's actual gait mechanics.

The monitoring devices can measure a user's locomotion events, such as steps and/or strides, which is used to identify the mechanical load experienced by the user. As used herein, a “step” refers to a locomotion interval associated with placement or movement of one foot relative to the other foot, whereas a “stride” refers to a locomotion interval extending between successive corresponding events of the same foot. A step is generally the interval from a contact event of one foot to the subsequent contact event of the other foot. A stride can be treated as the interval between successive initial contact events of a given foot and is complete when initial contact, midstance, toe-off, and a subsequent initial contact have been identified. As used herein, the mechanical load is different from the physiological effects (heart rate, respiratory rate, blood oxygen levels, etc.) experienced by the user and includes physical wear on a user's body (e.g., breakdown of the body tissues, such as muscle and tendon wear).

Each monitoring device can include internal sensors, such as an inertial measurement unit that detects acceleration and rotational movement, along with processing circuitry, memory, timing circuitry, a wireless communication arrangement, and a power source. In some implementations, each monitoring device operates in a plurality of operating states, such as an ultra-low-power sleep state, an idle state, and an active recording state. In the sleep state, the IMU can remain active at a reduced-power level and can wake other circuitry upon detection of motion satisfying one or more thresholds. In the idle state, the monitoring device can be available for connection to a host computing device, such as a smartphone, for exchange of status information, logs, stored workout data, or timing information. In the active recording state, the monitoring device can acquire inertial data at a higher sampling rate, such as about 480 Hz for example, and can process the inertial data in real time to generate stride-level data for local storage and optional streaming. This allows the system to work in a relatively unobtrusive manner, without requiring the user to manually start or stop each session.

The disclosed system collects data from both feet and later associates those two data sets with one another. This association can be based on timing information so that the system can determine how movements of the left foot relate to movements of the right foot over time. In practical terms, this enables the system to evaluate bilateral movement patterns (i.e., how the two sides of the body compare and interact during an activity). That is important because many gait problems, inefficiencies, and injury risks are not apparent from one side alone. In some implementations, bilateral association can include interpolating or otherwise mapping first-foot data and second-foot data onto a common time basis, such as a common one-second grid or other fused timing structure, to create a fused bilateral data set usable for downstream analytics. In some implementations, the first-foot data and the second-foot data include stride data records and associated timing information from which one or more step-related values, stance-phase values, swing-phase values, or other derived gait values are determined for downstream analytics.

The system can also analyze motion at a finer level by identifying particular events within each stride or stride-represented locomotion interval. For example, the system can identify when a foot first contacts the ground, when the foot leaves the ground, and the periods during which the foot is bearing weight or moving through the air. From those events, the system can determine gait-related measurements such as stride length, stride velocity, step frequency, stance time, flight time, swing time, foot strike pattern, pronation-related information, impact-related values, vertical oscillation asymmetries, and other movement features. Those measurements can be determined for individual stride data records, for groups of stride data records, for segments of a workout, or for an activity as a whole.

A resulting stride data record can include, for example, a timestamp for initial contact, a timestamp for midstance, a timestamp for toe-off, a pronation excursion value, a foot strike angle, an accumulated distance value, an accumulated velocity value, and an indication of left-foot or right-foot association. As used herein, “stride data” refers to processed, stride-level data derived from raw sensor data for a given stride and excludes the raw sensor data itself. In some implementations, stride data is stored as a single stride record including one or more processed values associated with that stride. In some implementations, local storage is organized as stride data records, while one or more step-related values, intervals, events, phases, or metrics, including stance-phase values or swing-phase values, are derived from the stride data records for downstream analysis. Accordingly, discussion herein of step-based, stance-phase-based, or swing-phase-based analysis does not require that local storage be organized as raw sample data or as separate step data records.

Another aspect of the system is that the devices can gather data locally during the activity and then transfer the data later to another computing device, such as a smartphone, smartwatch, tablet, or remote server. This means the system does not necessarily depend on a constant live connection during the activity itself. After data is uploaded, a host application and, in some cases, a remote computing system can perform additional analysis. For example, the system can correct for differences in timing between the two footwear devices, align the left-foot and right-foot data, refine the derived metrics, and compare current movement patterns against prior activities. In some implementations, the locally stored data is not a continuous raw-data record for the entire activity, but instead a local data set based on stride metrics obtained by real-time processing of raw inertial measurements. This can materially reduce memory usage relative to storage of continuous raw 480 Hz inertial samples, for example, thereby enabling storage of substantially longer activity records in-shoe. For example, the raw data sampling frequency can be one or more orders of magnitude greater than a stride frequency represented by the stride data records. In some implementations, workout data remains stored locally until upload to the host computing device has been completed successfully. In some implementations, the uploaded local data set includes stride data records and associated timing information, and one or more step-related values, stance-phase values, swing-phase values, or other downstream analytical values are derived after upload from the stride data records and associated timing information.

The system can also use this information to form a longer-term understanding of a person's movement. Over time, the system can build a baseline profile that represents the user's typical gait characteristics. New activity data can then be compared against that baseline to determine whether the user is moving in a way that differs from their ordinary pattern. The system can also calculate cumulative impact-related information across many steps, which can help indicate how much mechanical stress the user may be experiencing during a workout or over a period of training. In some implementations, historical stride data can additionally be compressed or summarized into a representative data set, such as by grouping strides into pace ranges or speed ranges and retaining a subset of stride records within each range, thereby preserving relationship information such as pace-versus-impact behavior while reducing storage and computational burden for downstream recommendation logic.

Using these analyses, the system can generate recommendations for the user. Those recommendations can relate to modifying gait, reducing impact, improving symmetry between the feet, performing suggested exercises, runs or workouts that are personalized to the user for improved performance and reduced injury risk, pacing, or taking some other action related to performance or recovery. The recommendations can be provided during the activity in real time or afterward as part of a summary or training report. They can be delivered through audio, visual, or other outputs on a connected computing device. In some implementations, the recommendation can be based on a cumulative impact metric, an impact score, a resiliency score, a baseline deviation, a bilateral symmetry metric, a pace-conditioned anomaly, or combinations thereof. In some implementations, the system additionally provides a running-signature output summarizing one or more user-specific gait tendencies and/or an efficiency score based on a relationship between a mechanical-output-related value and a physiological-cost-related value.

Overall, the system provides a more accurate and useful way to understand human movement by placing sensing technology at a known predetermined fixed position on the foot, associating data from both sides of the body, and converting that information into meaningful gait analysis and guidance. Although running is one example, the same approach can be applied in other contexts, including walking, sports training, rehabilitation, occupational monitoring, and other situations in which detailed understanding of foot motion and bilateral gait patterns may be valuable.

### Hardware System Overview

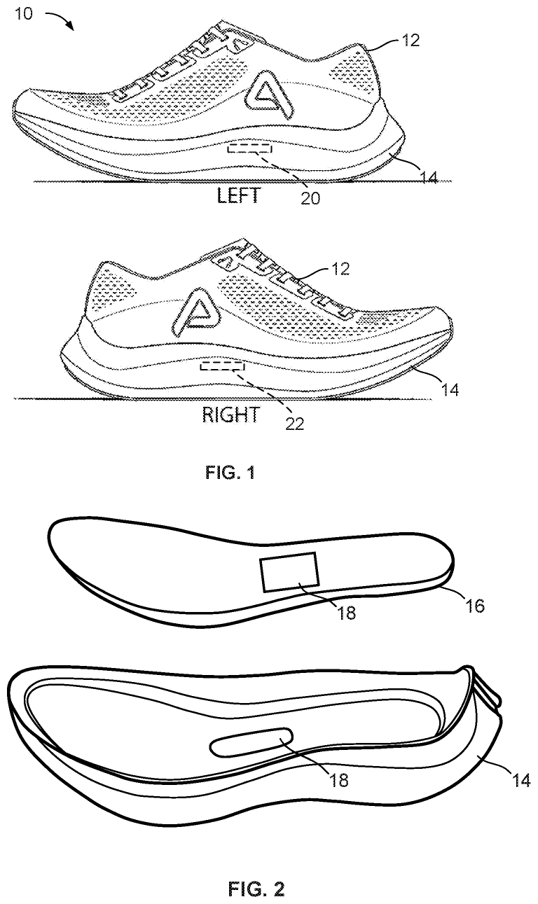

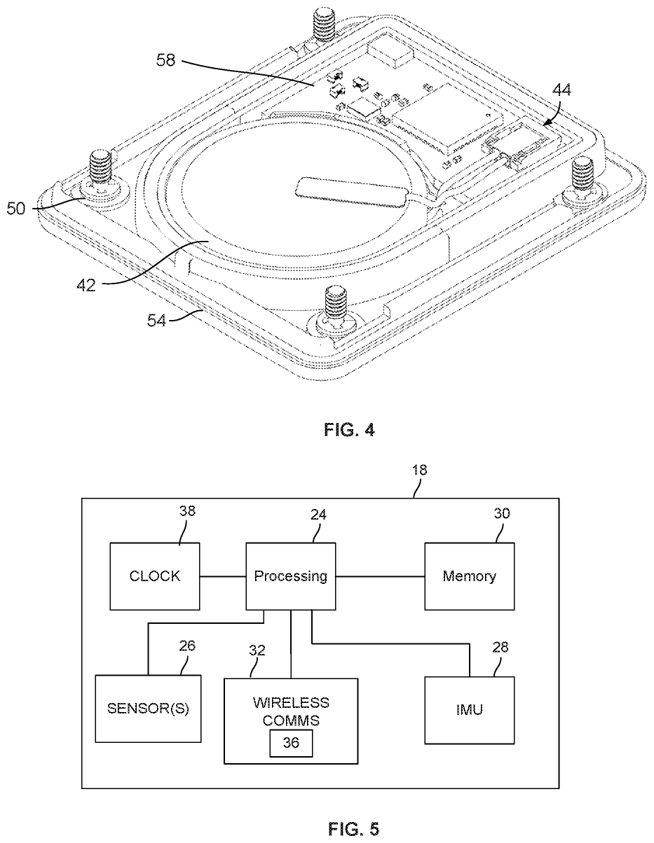

Referring to FIGS. 1-5, a footwear assembly 10 includes a footwear article 12 having a sole 14. The footwear article 12 can be configured to receive a monitoring device 18 below a foot of a user. More specifically, the monitoring device 18 can be positioned below a midfoot region of the user. The sole 14 can support an insole 16, and the sole 14 and/or the insole 16 can define or help define a cavity, recess, compartment, or other underfoot receiving region configured to receive the monitoring device 18.

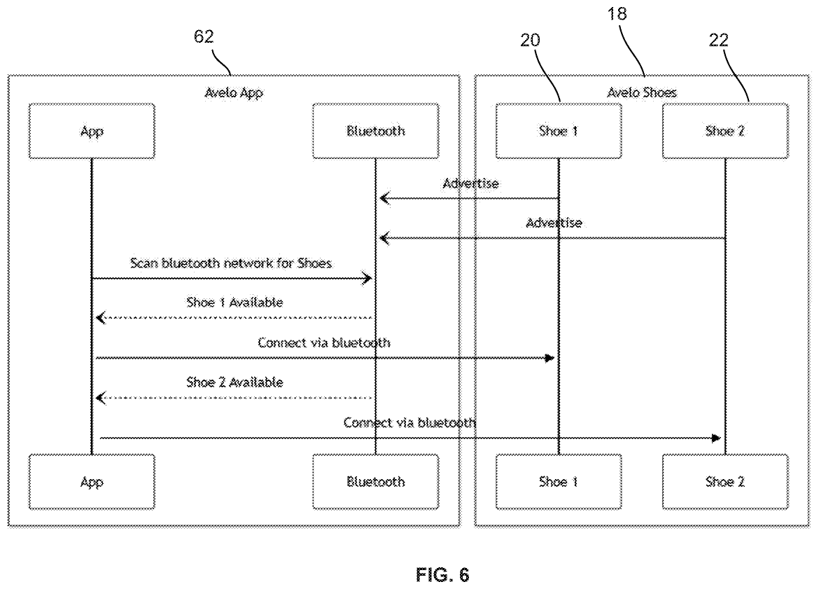

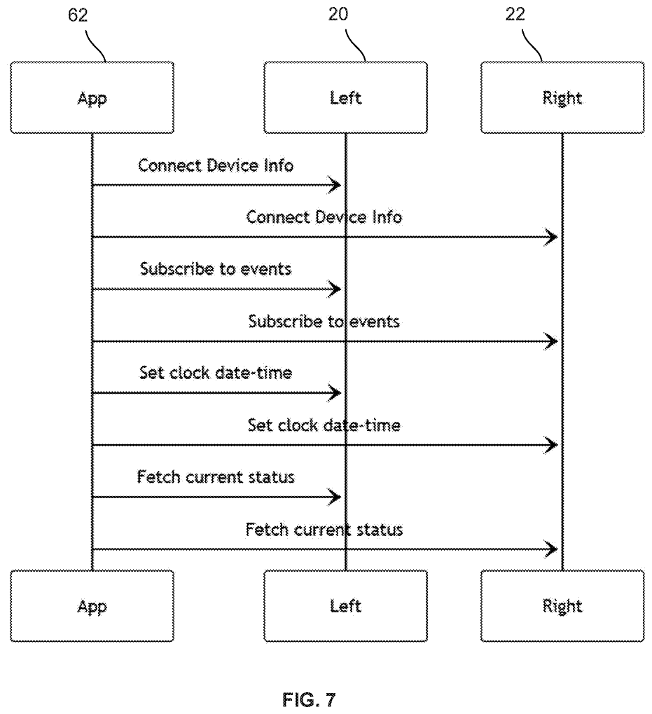

With additional reference to FIGS. 6 and 7, the monitoring device 18 can be implemented as a first monitoring device 20 associated with a first foot of the user and a second monitoring device 22 associated with a second foot of the user. In some implementations, the first monitoring device 20 and the second monitoring device 22 can be substantially identical and can operate as a bilateral monitoring system. In other implementations, the first monitoring device 20 and the second monitoring device 22 can differ in one or more respects, such as form factor, configuration parameters, stored identifiers, calibration data, firmware settings, wake thresholds, memory allocation, or communication behavior.

The first monitoring device 20 can include a first processor 24, one or more first sensors 26, a first inertial measurement unit (IMU) 28, first memory 30, a first wireless communication architecture 32, a first antenna 36, a first long-term power source 34, and optionally a first real-time clock 38. The second monitoring device 22 can likewise include a second processor 24, one or more second sensors 26, a second IMU 28, second memory 30, a second wireless communication architecture 32, a second antenna 36, a second long-term power source 34, and optionally a second real-time clock 38. The processor 24, memory 30, timing circuitry such as the real-time clock 38, and associated logic and circuitry used to acquire inertial data, store data, perform local processing, and communicate with an external computing device can collectively be characterized as a control module. The first long-term power source 34 and the second long-term power source 34 can power the corresponding monitoring devices 20, 22 without recharging for at least 100 hours of active running, at least 125 hours of active running, at least 150 hours of active running, or for a service life that exceeds an expected useful life of the corresponding footwear article 12.

Each of the first monitoring device 20 and the second monitoring device 22 can therefore serve as a separate computing device and control module associated with a respective footwear article 12 and configured to receive raw inertial data from a respective IMU 28, process the raw inertial data in real time during an activity to generate stride data, and store a local data set based on the stride data. The first monitoring device 20 can be secured relative to the first foot, or secured for movement with the first foot, and the second monitoring device 22 can be secured relative to the second foot, or secured for movement with the second foot, such that inertial measurements acquired by the respective monitoring devices 20, 22 correspond to movement of the respective feet during the activity. In some implementations, workout records can be stored sequentially and identified by respective headers including start-time information. In some implementations, running metrics can be stored as a millisecond offset from the beginning of a current run, with a timestamp taken at the beginning of the run.

In some implementations, the first processor 24 can be configured to control acquisition of inertial measurements from the first IMU 28, local processing of the inertial measurements, storage of locally derived stride data and associated workout records in the first memory 30, and communication of data using the first wireless communication architecture 32. The second processor 24 can be similarly configured with respect to the second IMU 28, the second memory 30, and the second wireless communication architecture 32. In some implementations, one or more step-related values, stance-phase values, swing-phase values, or other downstream analytical values are derivable from the stored stride data and associated workout records.

In some implementations, the first monitoring device 20 and the second monitoring device 22 can be designed and programmed to automatically capture events associated with an activity such as running without requiring intervention of the user. The first IMU 28 can automatically wake circuitry of the first monitoring device 20 upon motion exceeding one or more thresholds, and the second IMU 28 can automatically wake circuitry of the second monitoring device 22 upon motion exceeding one or more thresholds. Components of the first monitoring device 20 and the second monitoring device 22 can selectively operate as necessary for particular functions to reduce power consumption. The first antenna 36 and the second antenna 36 can be inactive during portions of data collection to reduce power consumption. In some implementations, the first monitoring device 20 and the second monitoring device 22 can record information about a run or other activity without user intervention and without requiring a third-party device during the activity. The independent functionality of the monitoring devices 20, 22 can allow each monitoring device to record running independently of a connection to a connected computing device such as a smartphone. In some implementations, each monitoring device 18 can transition among an ultra-low-power sleep state, an idle state, and an active recording state. In the sleep state, the IMU 28 can remain active as a wake source. In the idle state, the corresponding monitoring device 18 can advertise and accept a connection from a host computing device 62. In the active recording state, the corresponding monitoring device 18 can sample inertial data at a high rate, such as approximately 480 Hz, perform secondary run detection, generate stride data, optionally stream selected information to the host computing device 62, and store workout data locally. In some implementations, the bilateral system architecture can provide reduced user burden relative to body-worn rechargeable wearables because the monitoring devices 20, 22 can remain integrated with the footwear article 12, can initiate recording automatically without requiring a live connection to the host computing device 62, and can store workout information locally for later synchronization.

In some implementations, the bilateral architecture can be composed of two identical monitoring devices 18, each capable of detecting and recording running data independently and connecting independently to a connected external computing device 62, such as a smartphone. Any such monitoring device 18 can be used in either a left shoe or a right shoe. In some implementations, the side assignment can be determined dynamically from inertial data processed during running. This can simplify production, assembly, inventory control, servicing, and firmware distribution by avoiding separate left and right hardware versions. By using a single hardware design, the need for separate left and right SKUs can be eliminated, reducing inventory complexity. Final assembly can be simplified because any monitoring device 18 can be used in either the left or right footwear article 12 without pre-assignment. In some implementations, having only a single hardware design can mean that only a single version of firmware needs to be distributed, tested, and maintained, and that firmware updates can be deployed uniformly to all devices without concern for hardware variant compatibility. In some implementations, the side determination can be made algorithmically within the first few strides of a run without requiring user input, training, or pre-configuration, and with sufficient confidence that no manual override or correction is needed under normal use conditions.

In some implementations, because left/right assignment can be determined dynamically from the end user's data, the system can reduce the consequences of manufacturing or installation error and can simplify replacement and servicing. In some implementations, replacement monitoring devices 18 need not be side-specific, further simplifying servicing. In some implementations, the independent operation of the first monitoring device 20 and the second monitoring device 22 can also eliminate the need for routine device-to-device communication during recording, thereby reducing radio burden and preserving battery life. In some implementations, if one monitoring device 18 fails to record a segment or reports corrupted data, the data from the other monitoring device 18 can still be used to recover or estimate one or more values for the activity. In some implementations, data from a single functioning monitoring device 18 can be used to estimate certain metrics through inference from the available stride data, ensuring that a partial hardware or firmware failure does not result in complete data loss for a session. By computing derived stride metrics on-device rather than streaming raw inertial data, each monitoring device 18 can reduce wireless data transmission requirements, which can contribute to extended battery life. By having each monitoring device 18 independently compute stride data on-device, the need to stream or store raw IMU 28 data can be eliminated, which can significantly reduce power consumption.

In some implementations, other hardware arrangements can be employed. For example, there can be multiple monitoring devices 18 per shoe, multiple sensor locations within a shoe (e.g., a monitoring device 18 in a midfoot area and a monitoring device 18 in a forefoot area to detect differences between rearfoot and forefoot mechanics, with the additional monitoring devices 18 being mechanically independent or connected via a wired connection) pressure or force sensors in addition to the IMU 28, or asymmetric primary/secondary device architectures in which one monitoring device 18 receives data from the other monitoring device 18 and performs bilateral association locally. In a primary/secondary architecture, a primary monitoring device 18 can receive data from a secondary monitoring device 18 via a short-range wireless link and can perform bilateral association on-device, enabling real-time bilateral metrics without requiring a connected computing device 62 such as a smartphone. Further embodiments can incorporate additional sensors 26 at on-body locations such as a hip, wrist, head, chest, or other wearable location, including but not limited to chest-strap heart rate monitors, GPS-enabled watches, smart rings, and augmented-reality devices.

### Footwear Integration and Placement

The monitoring device 18 can be positioned within the footwear article 12 at a location arranged below the foot when the footwear article 12 is worn. The monitoring device 18 can be located within the sole 14, within the insole 16, between the sole 14 and the insole 16, or within another underfoot region of the footwear article 12. In some implementations, the monitoring device 18 can be positioned below a midfoot region, such as below an arch region or another underfoot location selected to balance signal quality, user comfort, structural protection, and communication performance. In some implementations, the first footwear article 12 and the second footwear article 12 can define corresponding receiving locations that promote comparable positioning of the first monitoring device 20 and the second monitoring device 22 relative to the respective feet, thereby improving bilateral comparison of measurements and derived gait metrics. In addition to running, the disclosed arrangement can be used to measure and monitor biomechanics in a variety of sports such as basketball, football, and soccer, as well as in walking, rehabilitation, and remote patient monitoring settings.

Positioning the monitoring device 18 below the foot can provide a stable relationship between the monitoring device 18 and foot motion during use and can facilitate repeated acquisition of inertial measurements associated with ground-contact events, stance-to-swing transitions, toe-off events, and other gait-related movement characteristics. Ground contact events can serve as primary temporal anchors for stride cycle segmentation. Because the position of the monitoring device 18 relative to the footwear article 12 can be fixed and known, the position information can also be used algorithmically to improve measurement quality.

In some implementations, the selected location corresponds to the medial longitudinal arch region of the foot, such that the monitoring device 18 can be aligned with the longitudinal axis of the foot while avoiding prominent bony structures at the plantar surface. In some implementations, the monitoring device 18 can be positioned inferior to the navicular and cuneiform region of the midfoot, where the plantar surface presents a natural recess that accommodates the monitoring device 18 with reduced risk of creating pressure points.

Measuring at the foot can enable direct detection of ground-contact and toe-off events from inertial signatures generated at the point of foot-ground interaction, rather than inferring such events from attenuated signals measured remotely from the foot. This can improve determination of stance phase, swing phase, and corresponding transitions. The foot can serve as the end-point effector for movement during running, and the motions and vibrations experienced at the foot can enable more accurate measurement of running gait than measurements taken at locations remote from the foot. The fixed and repeatable in-shoe location can also improve intra-runner, inter-runner, and bilateral comparisons because differences in measured metrics can more confidently be attributed to gait mechanics rather than to differences in sensor placement or sensor movements. Because both monitoring devices 20, 22 can occupy identical positions relative to their respective feet, bilateral asymmetry measurements can reflect actual left-right gait differences rather than artifacts of inconsistent sensor placement, which can be of particular benefit in clinical applications such as injury assessment and rehabilitation monitoring.

In one example embodiment, the monitoring device 18 can be fitted within a corresponding cavity in the midsole material just beneath the sock liner, such that the upper surface of the monitoring device 18 can be flush with or below the top surface of the midsole. This arrangement can maximize user comfort while still allowing insertion and removal of the monitoring device 18. The cavity can also ensure that the monitoring device 18 does not vibrate or move independently from the footwear article 12, such that the measured signals correspond to the true motion of the foot and footwear. In some implementations, placement in the heel can be avoided because such placement can interfere with cushioning, energy return, and stability of the footwear article 12. Placement in the forefoot can be avoided because such placement can interfere with the flex point of the footwear article 12 and can alter natural toe-off mechanics.

The selected in-shoe location can provide benefits over upper-body, ankle-mounted, or on-shoe alternatives. In contrast to wrist, chest, or hip-mounted devices: (a) the underfoot location can measure signals at the endpoint of the locomotor chain rather than at a location remote from the point of limb motion where signals can be diminished; (b) signals at upper-body locations can be dampened due to the mitigating effects of other joint mechanics such as the knee and hip; (c) upper-body locations can subject the monitoring device 18 to motion artifacts from inertial lag, where independent motion of the sensor itself can contaminate the signal rather than reflecting the true motion of the limb being measured; and (d) upper-body locations can be unable to capture specific foot movements such as pronation, foot strike pattern, and impact angle, which can only be measured at the foot. In contrast to ankle-mounted devices, the underfoot location can reduce placement variability because ankle straps or bands can shift position during activity, introducing measurement variability between sessions.

Ankle-mounted devices can be subject to soft-tissue motion artifacts because the strap can move independently of the underlying skeletal structure. Ankle-mounted devices may also be unable to directly measure foot-ground interaction events with the same fidelity as an underfoot sensor. In contrast to on-shoe devices, the underfoot location can reduce inertial lag because on-shoe attachment mechanisms can allow the device to move semi-independently from the shoe structure during high-impact events such as foot strike. On-shoe devices can be subject to repositioning by the user between sessions, introducing placement variability that can contaminate measurements. On-shoe devices may also be exposed to environmental damage, detach during activity, and alter the external profile of the footwear article 12. In-shoe placement can reduce visible bulk and the risk that the user forgets to wear or attach the device. Forgetting to wear or attach a strap-on or clip-on device can be a common failure mode that leads to incomplete data collection over time, and in-shoe placement can eliminate this failure mode because the monitoring device 18 can always be present when the user wears the footwear article 12. In-shoe placement can further shield the monitoring device 18 from external impacts and abrasion and can eliminate visible external hardware.

In some implementations, the monitoring device 18 can be permanently integrated into the footwear article 12, semi-permanently installed, removably received, or incorporated into an insert, insole 16, sock liner, midsole structure, orthotic, or other footwear component. The monitoring device 18 can be integrated into therapeutic or rehabilitation footwear for post-surgical recovery monitoring, enabling clinicians to track gait symmetry and loading progression during a patient's return-to-activity program. The monitoring device 18 can be incorporated into a disposable or single-use insole insert for clinical gait assessment, sports performance evaluation, or short-term biomechanical studies. The monitoring device 18 can be provided in a demonstration configuration in which sensor-equipped footwear is used for in-store or in-clinic gait analysis, with the monitoring devices 18 being transferred between multiple pairs of footwear. The monitoring device 18 can be integrated into custom orthotic devices, enabling ongoing monitoring of gait characteristics for patients with prescribed orthotic interventions. The monitoring device 18 can be integrated into military or tactical boots for load-carriage monitoring, fatigue detection, and injury-risk assessment. The monitoring device 18 can be integrated into occupational or industrial footwear for workplace ergonomics monitoring, fatigue tracking, and compliance with occupational health and safety protocols.

### Monitoring Device Assembly and Enclosure

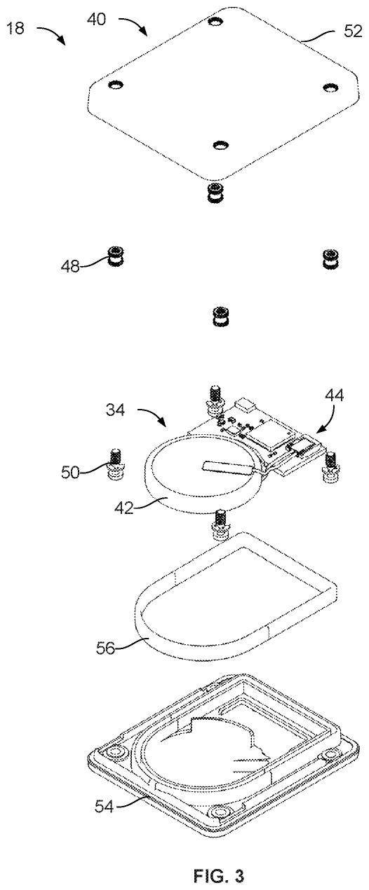

Referring to FIG. 3, each monitoring device 18 can include an enclosure 40. The first monitoring device 20 can include a first enclosure 40, and the second monitoring device 22 can include a second enclosure 40. Each enclosure 40 can be entirely self-contained and sealed, and in some implementations can be hermetically sealed. The exploded view of FIG. 3 illustrates a battery 42 forming the long-term power source 34, a battery connector 44, a threaded insert 48, a screw 50, a front shell portion 52, a rear shell portion 54, a seal ring 56, and a circuit board 58. One or more alignment structures can orient the monitoring device 18 relative to the footwear article 12 and can reduce rotational play, translational play, or inconsistent placement of the monitoring device 18 within the footwear article 12.

In one implementation, the enclosure portions 52, 54 can be injection molded from PA66+30GF (polyamide 66 reinforced with 30% glass fiber). This material can provide high impact resistance under repeated foot-strike loading, dimensional stability across the operating temperature range, resistance to moisture and sweat exposure, and a stiffness-to-weight ratio that enables thin wall sections without sacrificing structural integrity. The 30% glass-fiber reinforcement can provide improved rigidity and creep resistance compared to unfilled nylon, which can be important for maintaining seal compression and consistent enclosure geometry over the service life of the monitoring device 18 under cyclic loading. The material can also be sufficiently transparent to the BLE antenna signal for underfoot communication. Exterior surfaces of the enclosure 40 can optionally include printed or engraved markings, including orientation markings, serial numbers, regulatory marks, or codes.

In one example embodiment, the enclosure 40 can have a rectangular footprint with external dimensions in a range of approximately 30 mm to 45 mm in length, approximately 25 mm to 40 mm in width, and approximately 4 mm to 8 mm in height, the dimensions selected to accommodate the range of midfoot cavity sizes corresponding to the range of target shoe sizes while maintaining a profile sufficiently low that the monitoring device 18 is not perceptible to the user underfoot during use. In a specific example embodiment, the enclosure 40 can have approximate external dimensions of 37.2 mm by 32.6 mm by 6.0 mm. The enclosure geometry can be selected to balance several competing requirements: (a) sufficient internal volume for the battery and electronics; (b) a profile low enough to fit within the midsole without being perceptible to the user; (c) a footprint small enough to fit within the midfoot region of the smallest target shoe size; (d) sufficient structural rigidity to survive repeated impact loading without flexing or cracking; and (e) an external geometry compatible with the alignment structures that orient the monitoring device within the midsole cavity. The rectangular form factor and corner fastening pattern can also provide uniform clamping force around the perimeter of the seal ring.

The enclosure 40 can include a perimeter groove for the seal ring 56, a recessed area for circuit-board mounting, a battery groove for receiving the battery 42, and screw bosses or corresponding fastening features. In some implementations, the enclosure wall thickness can be approximately 0.4 mm, with ribs or gussets added for stiffness, battery support, and compression relief. The top and bottom enclosure portions 52, 54 can mate along a perimeter flange, and the seal ring 56 can be compressed between them to achieve ingress protection, such as IP67 or IPX7 performance in some embodiments. The seal ring 56 can be designed to maintain integrity across repeated battery replacement cycles, such that each time the enclosure 40 is opened and resealed, the seal ring 56 can re-seat in its groove and restore the ingress barrier. In some implementations, the front shell portion 52 and the rear shell portion 54 can cooperate with the seal ring 56 to define a sealed internal volume containing the circuit board 58 and other internal components.

Prior to assembly, the circuit board 58 can be coated with conformal coating except at designated battery terminal or connector regions. The conformal coating can provide additional moisture, chemical, mechanical, and environmental protection for the electronics within the sealed enclosure 40. The circuit board 58 can be attached to the enclosure 40 by adhesive, such as an EVA-based double-sided adhesive, which can reduce space and weight relative to mechanical retention methods and can also provide vibration damping between the circuit board 58 and the enclosure 40, reducing the potential for high-frequency mechanical noise to contaminate the IMU 28 measurements.

The antenna 36 can be implemented as a chip antenna positioned on an outer edge of the circuit board 58. The antenna 36 can be positioned on the circuit board 58 at a location that maximizes distance from the battery 42, which can be the largest metallic component within the enclosure 40 and could otherwise detune or shield the antenna 36. The enclosure material and thickness in the antenna region can be selected to avoid undue attenuation of the RF signal.

In one implementation, the monitoring device 18 can be assembled as follows. An EVA adhesive can be applied into a groove on the interior surface of the front shell portion 52 and inspected for smooth, flat adhesion without warping. The seal ring 56 can be inspected for damage and installed into the perimeter groove of the front shell portion 52, ensuring it is seated flat and without warping. Screws 50 can be placed into screw holes at the four corners of the front shell portion 52, and corresponding retention features can be attached below the screw threads. The circuit board 58 can be inspected for defects and both sides can receive conformal coating, excluding the battery connector region, using an anti-static brush. The EVA adhesive backing can be removed and the circuit board 58 can be attached to the interior of the front shell portion 52, pressed flat without warping. The battery connector 44 can be inserted into the terminal block on the circuit board 58, and the battery 42 can be placed into the battery groove of the front shell portion 52, with connector wires arranged so as not to be pressed under the battery 42, circuit board 58, or enclosure portions 52, 54. The rear shell portion 54 can be inspected and placed onto the front shell portion 52, with the battery connector wires confirmed not to be pinched between the enclosure portions 52, 54. The screws 50 can be driven at controlled torque at each corner position, securing the rear shell portion 54 to the front shell portion 52 and compressing the seal ring 56. The assembled monitoring device 18 can be laser engraved on the rear shell portion 54 with applicable regulatory identifiers, serial number, QR code, and product information. Visual inspection can be performed, including confirmation of no internal rattling and alignment of the enclosure joint. Functional testing can be performed using BLE-connected test software, including device scan, connection, serial number programming, self-test, battery level verification, and shipping mode activation.

### Power Source and Retention Arrangement

In some implementations, the battery 42 can be a generally circular coin-cell battery, such as a CR2032 battery having a nominal capacity of about 220 mAh. A long-life primary cell can be selected to provide sufficient energy density within the geometric constraints of the enclosure 40 and to support a service life that can be at least comparable to, or greater than, the useful life of the footwear article 12. In some implementations, the system can be designed to provide at least 100 hours of active running, and more specifically at least 125 hours of active running, on a single battery 42. In some implementations, the battery life target can be derived from a target shoe service life and an assumed average running pace, such that the battery 42 does not become the limiting factor in the useful life of the monitoring device 18. When not recording a run, the monitoring device 18 can enter a deep sleep state that draws minimal current, thereby preserving battery capacity for active use.

In one implementation, the battery retention arrangement can use a fully wired connection between the battery 42 and the circuit board 58. Positive and negative tabs can be connected directly to the battery 42 and coupled through wires to a connector, such as a Molex connector, on the circuit board 58. This arrangement can improve reliability under repeated impact and vibration by avoiding intermittent disconnects that can occur in spring-contact battery arrangements. The fully wired connection can prevent loss of battery connection during use.

The battery connector 44 and surrounding enclosure features can cooperate to reduce battery movement and maintain reliable electrical continuity. In some implementations, the battery 42 can be replaceable by opening the enclosure 40, disconnecting the battery connector 44, replacing the battery 42, and resealing the enclosure 40. In one example, the battery replacement procedure can be completed using a small screwdriver, such as a T4 driver, without specialized equipment.

Alternative power-source configurations can also be used. For example, a rechargeable battery, a wireless-charging configuration, contact-based charging pads, or other energy-storage arrangements can be employed, with corresponding adaptations to the power-management subsystem and enclosure design.

In some implementations, the firmware can monitor battery voltage during operation and can log the battery status with each measurement cycle. The monitoring device 18 can additionally estimate remaining battery capacity based on the monitored battery voltage or other battery-related information. When data is uploaded to the host application, the application can review the battery logs and the estimated remaining capacity and can inform the user if the voltage and/or estimated remaining capacity has fallen below a threshold, thereby providing advance notice that the battery 42 should be replaced.

### Orientation, Calibration, and Side Determination

One or more alignment structures can cooperate with corresponding structure of the footwear article 12 to orient the monitoring device 18 in a predetermined orientation relative to the foot. Features can be utilized to place each of the first and second monitoring devices 20, 22 within the footwear articles 12 to provide proper orientation along and about each of an x-axis, y-axis, and z-axis, and for the left or right foot as appropriate. The alignment structures can include asymmetrical outer surfaces, keyed features, protrusions, recesses, tabs, ribs, rails, chamfers, mating interfaces, or graphical markings indicating installation direction. The predetermined orientation can align one or more sensing axes of the IMU 28 with one or more anatomical axes of the corresponding foot.

Providing a predetermined orientation can improve manufacturability, user installation reliability, and algorithm performance. In some implementations, the IMU signals can be used without applying scaling or offsets to correct for sensor bias, because drift correction can be handled by on-board algorithms that exploit the predetermined orientation. Correcting sensor bias may otherwise require sophisticated bench-top testing methods and equipment during production, which can add significant time and cost. By relying on the predetermined orientation to support on-device drift correction, factory calibration of sensor bias can be avoided.

Because the drift correction can be applied using a known reference orientation rather than an estimated one, the correction itself can be more accurate, and the corrected signals can more closely reflect the true motion of the foot. Over the course of a run comprising many strides, these per-stride accuracy improvements can accumulate into meaningfully more reliable derived metrics compared to a system that estimates its own orientation from the data. Without the predetermined orientation, additional data would need to be collected during stance to resolve sensor position relative to gravity before secondary drift corrections could be applied, which can be particularly challenging at higher running speeds where stance duration decreases and accumulated errors increase.

In some implementations, left/right recognition can also be based at least in part on the predetermined orientation. In some implementations, the recognition can be provided by known differences expected between the information generated from the left and right IMU 28. No training data is required for this determination. In some implementations, the host application can also verify expected IMU signals when the devices are connected and can alert the user if the monitoring device 18 has been installed in an incorrect orientation. In other embodiments, a fixed installed orientation need not be required. Instead, the monitoring device 18 can self-calibrate on wake-up or at another suitable time to identify proper orientation information, and can determine orientation information from measured characteristics of orientation and usage.

### Environmental, Durability, and Mechanical Performance

The enclosure 40 and associated structure of the monitoring device 18 can be designed to withstand repeated impact, vibration, moisture exposure, and temperature variation encountered during running and other activities. In some implementations, the monitoring device 18 can weigh less than approximately 15 grams including the battery 42, and more specifically approximately 10 grams or less. In some implementations, a weight of approximately 10 grams can represent approximately 3% of a typical running shoe weight, which can place the monitoring device 18 below a threshold at which runners can perceive an in-shoe device during use. Maintaining the monitoring device 18 below this perceptibility threshold can be a deliberate design objective, because a perceptible device may discourage use and can render the system impractical regardless of its measurement capabilities. The operating temperature range can be about −5 °C to 45 °C, and the survival temperature range can be about −25 °C to 80 °C. In some implementations, the assembled monitoring device 18 can be configured to satisfy an ingress-protection rating such as IP67 or IPX7.

In some implementations, the monitoring device 18 can be designed to exceed the useful life of a footwear article 12, such as a 500-mile or 800-kilometer shoe life, and can be transferred between successive pairs of footwear. In some implementations, the monitoring device 18 can be intended to be transferred between two successive pairs of footwear articles 12.

The monitoring device 18 can be inserted into the midsole cavity from above after removal of the sock liner. The cavity can be shaped to receive the enclosure 40 in a single orientation. Once seated, the monitoring device 18 can be retained by close-tolerance friction fit, downward compression from the sock liner, overlapping structural flaps, or combinations thereof. The midsole material surrounding the cavity can conform slightly to the enclosure walls, damping any residual micro-movement of the monitoring device 18 relative to the footwear article 12. In some implementations, the cavity dimensions can be toleranced to reduce translational and rotational movement, thereby ensuring that inertial measurements correspond to the true motion of the foot and footwear rather than to independent device motion. In some implementations, one or more anti-rattle features, anti-rotation features, compliant inserts, ribs, pads, keyed surfaces, or other retention structures can be provided to further inhibit movement of the monitoring device 18 relative to the footwear article 12 during use. Alternative retention approaches can include snap-fit retention, adhesive retention, cartridge or drawer insertion, molded-in-place retention, and sock-liner integration.

### Electronics and Wireless Communication

The circuit board 58 can support the processor 24, sensors 26, IMU 28, memory 30, and circuitry of the wireless communication architecture 32. The processor 24 can include any suitable processing unit or combination of processing units with radio-frequency communication capability configured to perform wake-up operations, sensor processing, communication operations, power management, and memory management. An example processor can include any suitable processing unit or combinations of processing units (e.g., an RF-enabled microprocessor unit) to perform the necessary functions of the monitoring device, such as performing the wakeup routine, processing data from the IMU, initiating communication sessions, monitoring the battery operation and life, managing the available memory on the monitoring device, and performing RF communications. Memory 30 can be any suitable memory unit including RAM, ROM, EEPROM, or any other suitable memory types and combinations thereof. A clock of the processing unit, or in some embodiments a separate clock or oscillator, can be utilized for synchronization of data between monitoring devices and with third-party devices, thus avoiding or limiting timing mismatches such as due to frequency drift. Accordingly, the clock timing and frequency can be modified and/or updated to allow for phase and offset correction.

In some implementations, temperature sensing can be available from the IMU 28, the processor 24, or both, and temperature values can be logged during operation. Battery-voltage monitoring can also be performed and logged. In some embodiments, additional sensors 26 can be incorporated, such as pressure sensors, force sensors, barometric sensors, magnetometers, or other sensors. Output components, such as a haptic actuator, can also be included.

The wireless communication architecture 32 can include Bluetooth Low Energy circuitry, near-field communication circuitry, proprietary short-range radio circuitry, Wi-Fi circuitry, or combinations thereof. The antenna 36 can be positioned and tuned for communication from the underfoot location to an external computing device.

The real-time clock 38 can be used to generate or maintain timing information associated with acquired inertial data, locally derived gait metrics, stored run records, or combinations thereof. In some implementations, the host computing device 62 can periodically update the real-time clock 38 while the monitoring device 18 is in the idle state, and not while the monitoring device 18 is actively recording, syncing, or uploading, to reduce accumulated drift and restore accurate time after a power interruption.

The timing circuitry need not, by itself, maintain complete bilateral synchronization between the first monitoring device 20 and the second monitoring device 22 during the activity. Instead, the timing circuitry can provide timing information that can be sufficiently precise to support later association and correction of data sets acquired at the respective monitoring devices 20, 22. In some implementations, post-processing bilateral synchronization can include a coarse alignment determined at the host computing device 62 based on run-start timing, real-time clock 38-derived timing information, or both, followed by a finer alignment determined after upload using detected step events, contact events, segmented drift estimation, or combinations thereof. Step events can serve as alignment anchors, particularly where the respective monitoring devices 20, 22 began recording at different times or where timing confidence varies across an activity. In some implementations, initial timing association can be performed at the host computing device 62 using a system clock of the host computing device 62 together with timing information received from the first monitoring device 20 and the second monitoring device 22, while additional refinement of offset correction, drift correction, phase correction, or other bilateral alignment can be performed at the remote server 64 after upload. In some implementations, the finer alignment can include interpolating first-shoe and second-shoe time streams, including contact-event timing, onto a common time grid using linear interpolation to generate a fused bilateral data set.

### Wake Logic, Power Modes, and Local Storage

The monitoring device 18 can have a plurality of power states configured to maximize battery life while enabling automatic activity detection and recording. In some implementations, the monitoring device 18 can enter a deep sleep state after a defined timeout period of inactivity of approximately five minutes, for example. In the deep sleep state, the main processor 24 and radio can be powered down while the IMU 28 can remain active in a low-power wake-on-motion mode. When the IMU 28 detects motion exceeding a threshold, a hardware interrupt can wake the main processor 24 and can transition the monitoring device 18 to an idle state. In some implementations, the IMU 28 can monitor accelerometer output at a reduced sample rate of about 1.875 Hz, for example, in the deep sleep state and can generate a hardware interrupt when the measured acceleration exceeds a motion threshold, with the wake-on-motion detection running entirely on IMU 28 hardware while the main processor 24 remains powered off. Thresholds used for wake-up can be dynamically selected for the user and/or modifiable by the user.

Upon entering the idle state, the monitoring device 18 can enable an embedded classification function, such as a machine-learning core or state machine within the IMU 28, to distinguish among motion classes such as inactivity, walking, and running.

When the classification logic indicates running, the monitoring device 18 can transition to an active recording state. During the transition, the IMU 28 can be reconfigured to sample the accelerometer and gyroscope at full data rate, and the firmware can begin processing stride data and writing metrics to memory 30. In some implementations, a confirmation period can be used to verify sustained running activity before a full recording session can be committed. If the activity ceases during the confirmation period, the monitoring device 18 can return to the idle state without creating a recording.

Once recording has started, the monitoring device 18 can continue to record stride-by-stride running metrics until the activity stops. When the firmware detects cessation of running, such as based on absence of detected strides for a defined timeout period of approximately five minutes, the current recording segment can be closed and the monitoring device 18 can return to the idle state. If inactivity continues for a further threshold period, the monitoring device 18 can return to the deep sleep state. In some embodiments, walking intervals can be not recorded, while in other embodiments the system can continue recording through walk intervals or record walking as a separate data type.

In some implementations, each monitoring device 20, 22 can perform wake detection and activity classification independently, without inter-device communication during the wake or recording process. The first monitoring device 20 and the second monitoring device 22 can wake independently and can later be associated based on timing information and detected step events. Accordingly, one of the first monitoring device 20 or the second monitoring device 22 can begin acquisition before the other, can continue acquisition after the other stops, or can otherwise acquire data over only partially overlapping time periods, while the resulting data sets can still be later associated using the timing information, detected event timing, and/or one or more correlation parameters. In normal use, both monitoring devices 20, 22 can detect the onset of running and can begin recording within effectively the same stride cycle. Any small timing differences between the two recordings can be resolved during later post-processing.

In some implementations, a race mode can be activated through the host application. In race mode, one or more activity-classification thresholds can be bypassed, and the monitoring device 18 can begin recording upon detection of motion without waiting for full confirmation of sustained running activity, eliminating latency between the start of an activity and the beginning of recording to ensure that the first strides are captured. Race mode recording can continue until the user manually stops the recording through the application or until a maximum duration timeout is reached. In some implementations, activity classification thresholds can be dynamically adjusted based on the user's historical running characteristics or manually adjusted through the application.

Once the monitoring device 18 has transitioned into the active recording state, the processor 24 can calculate and can store stride data in memory 30 in real time without storing the raw IMU data. The stride data can include, for example, timestamps for initial contact, midstance, and toe-off together with pronation excursion, foot strike angle, stride length, stride time, accumulated distance, accumulated velocity, underlying acceleration and angular velocity values, and side information. Rather than storing the full stream of raw 480 Hz inertial samples for an activity, the processor 24 can store a local data set based on stride data, thereby materially increasing in-shoe storage capacity. Storage of raw continuous inertial data would permit only a relatively short recording duration, whereas storage of processed stride data can permit storage of workout information for a substantially longer period, including on the order of a day or more of running data. Raw and intermediate per-sample processing values can be discarded after a stride has been completed and the corresponding stride metrics have been retained, thereby improving memory efficiency. Storage in memory 30 can be organized such that activity data for a substantial number of running hours can be retained locally until upload.

In some implementations, the processor 24 can additionally determine one or more of step rate, step time, stance time, swing time, flight time, contact time, vertical oscillation, foot orientation, cadence, and other impact-related values. In some implementations, values including duty factor, spring ratio, leg spring stiffness, braking forces, path of motion, turn-related information, and bilateral or symmetry-related values can be derived at the host computing device 62 or remote server 64 from the uploaded stride data, as described further below. Pronation excursion and foot strike angle can be determined on-device because those values can depend on raw signals that are not retained after stride compression. The on-device stride metrics can be transmitted to the host computing device 62 at an effective data rate corresponding to stride frequency rather than raw IMU 28 sample rate. Additional derived metrics, such as stride duration, stance duration, swing duration, flight duration, step duration, cadence, duty factor, spring ratio, and ground-reaction-force-related values, can then be determined at the host computing device 62, a remote server 64, or both, based on the uploaded stride data. In some implementations, the firmware can support per-step calculations of each of the above metrics in addition to averages for the entire run.

Computational allocation can vary. For example, event detection, pronation-related calculations, foot-strike-angle calculations, and stride-data generation can be performed on the monitoring device 18. Bilateral pairing, workout reconciliation, pacing summaries, split views, and selected coaching outputs can be performed at the host computing device 62. Historical aggregation, representative-data-set generation, impact-score normalization, resiliency-score determination, anomaly detection, machine-learning operations, and recommendation ranking can be performed at the remote server 64. In other embodiments, one or more such operations can be reallocated among the monitoring device 18, the host computing device 62, and the remote server 64 according to available power, memory, latency, privacy, or communication constraints. In some implementations, based on available processing and memory of the host computing device 62, some or all operations described as being performed at the remote server 64 can be performed locally at the host computing device 62, such as determining derived metrics, executing machine learning algorithms, creating custom scores, or creating training programs.

In a bilateral configuration, the first monitoring device 20 and the second monitoring device 22 can each store local data records and corresponding timing information independently during an activity. Memory 30 can store sequential workout records and corresponding headers including start times. In some implementations, if workout memory 30 reaches capacity, the monitoring device 18 can stop recording new workouts until data has been uploaded and memory 30 cleared, rather than overwriting the oldest stored workout data. In contrast, device logs can be permitted to overwrite older log entries when log storage reaches capacity. In some implementations, a workout can be treated as complete only after a threshold non-running interval has elapsed following apparent cessation of running. If the user pauses for more than approximately one minute but less than approximately ten minutes, the current workout segment can be closed and a new segment header can be created within the same workout record when running resumes. If the user pauses for more than approximately ten minutes, the current workout can be closed and a subsequent running period can be stored as a new workout record.

In some implementations, locally stored data from the first monitoring device 20 and the second monitoring device 22 can be retained until both monitoring devices 20, 22 have connected to the host computing device 62, uploaded their respective data, and confirmed successful transfer and bilateral reconciliation. Memory clearing can be deferred until sufficient bilateral upload and timing reconciliation has been completed. This can reduce the risk that unilateral deletion of data from one monitoring device 18 impairs later association of first-foot and second-foot data.

### Pairing, Initialization, and Synchronization

Referring to FIG. 6, a host computing device 62 can execute an application that communicates with the first monitoring device 20 and the second monitoring device 22. One or more remote servers 64 can communicate with the host computing device 62. The host computing device 62 can be a smartphone, smartwatch, tablet, laptop, desktop computer, or other host device. In an example embodiment, each of the first and second monitoring devices 20, 22 can send a BLE advertising message at predetermined intervals or in response to being woken by motion. An application executing on the host computing device 62 can scan for the first and second monitoring devices 20, 22 based on known identifiers or other values and can identify and display the first and second monitoring devices 20, 22 within the application. The application can then pair and connect with each monitoring device 18 using a suitable protocol such as BLE, for example based on a user selection or automatically under certain conditions, such as when only two monitoring devices 18 are in proximity to the host computing device 62.

Referring to FIG. 7, upon establishing a paired connection, the application can initialize the first and second monitoring devices 20, 22 for operation by creating a virtual connection between the first and second monitoring devices 20, 22, establishing initial device information by fetching current device status from each monitoring device 18, setting the real-time clock 38 of each monitoring device 18 independently by setting the clock date-time on each device, subscribing to events for exchanging information between the monitoring devices 20, 22 and the application, and connecting device information for downstream identification. Such events can include battery level updates, status updates, new run availability, inertial measurements exceeding thresholds, falls, ground force thresholds, and changes in stride or gait. In some implementations, pace or race time information can be collected from the user during initial setup to support pace zone determination as described further below. In certain modes of operation, parameters determined at the monitoring devices 20, 22 can be made available at update rates on the scale of milliseconds or seconds for display at the host computing device 62.

The firmware of the first and second monitoring devices 20, 22 can be updated by the application via the wireless link. The application can display running metrics for each split of a run or show how each metric changed throughout the run. The above metrics can be available in real time via the wireless link, with the update rate at least partially dependent on battery life requirements. In some implementations, the host computing device 62 can serve as a contact point for both monitoring devices 20, 22 and can combine uploaded stride data into a paired-shoe record for downstream analytics.

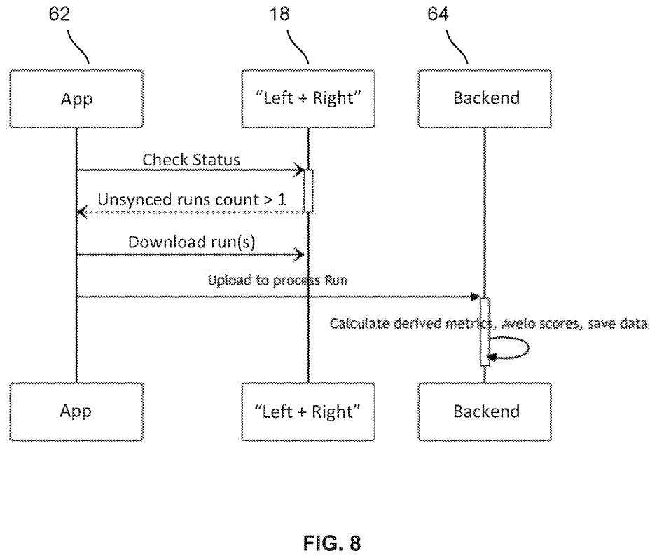

Referring to FIG. 8, when the application connects to the first and second monitoring devices 20, 22, the application can check the status of each monitoring device 18 and determine whether there is run data available for upload by checking whether the count of un-synced runs is greater than one. If run data is available, the data for each run, including underlying segments, can be provided by each monitoring device 18 to the application and downloaded to the host computing device 62. Data from both monitoring devices 20, 22 can be obtained and reconciled for timing, frequency, and data integrity, and the reconciled data can be uploaded to the remote server 64 for processing. The remote server 64 can calculate derived metrics, scores, and save the resulting data. Once runs are successfully downloaded from both monitoring devices 20, 22, the monitoring devices 20, 22 can be informed of the successful synchronization and can clear the corresponding memory locations. In some implementations, the application can request uploads from the oldest workout forward and can refrain from requesting upload while a corresponding monitoring device 18 is actively recording.

The application can utilize local processing to perform further calculation or manipulation of running metrics and can augment the running metrics with other data, such as heart rate, blood oxygenation, weather, climate, altitude, pressure, sleep, VO2max, body temperature, hydration levels, lactic thresholds, GPS or other position data, or self-reported information such as height, weight, injury history, and phone or other location data. In some implementations, the host computing device 62 can combine first-shoe stride data and second-shoe stride data into a shoe-pair representation that can then be uploaded to the remote server 64 for further analytics.

In some implementations, the remote server 64 can perform further analysis including machine learning or artificial intelligence processing and recommendations. Based on appropriate user permissions, metrics, derived values, and other information can be stored for a user and collated and compared between activities and between users to establish and update personal scores and recommendations. For example, impact force data for a user can be used to determine instantaneous and cumulative impact scores representing the overall mechanical load of a run for the purpose of optimizing training and recovery. In some implementations, the remote server 64 can additionally determine a representative data set from historical stride data by grouping strides by pace or speed range and retaining a subset of stride records within each range for later modeling of pace-versus-impact or other user-specific relationships. Derived values such as impact scores can be used to provide personalized recommendations including personalized running programs, running style recommendations, potential injury warnings, deviation alerts, and unique scores such as an impact score, resiliency score, and efficiency score. When the application is operating on the host computing device 62 during an activity, audio or other real-time recommendations can be provided based on real-time data, derived values, and combinations thereof. In some implementations, the application can provide real-time coaching based on cadence or step frequency, for example by a metronome output, and can additionally or alternatively provide real-time coaching based on impact-related values, efficiency-related values, bilateral asymmetry, detected anomalies, or pace-conditioned baseline deviations.

In some implementations, completed running files can be uploaded from the monitoring devices 20, 22 to the host application and then to a backend system. After confirmed backend storage, the local files can be cleared or overwritten. In some implementations, the backend can treat the two uploaded files from the same user account as a left-right pair for the same activity. In some implementations, no serial number, MAC address, or other hardware identifier need be used in the pairing logic, and a Bluetooth identifier can be stored with the data without being used for pairing or side determination.

### Analytics Processes

While the processes described herein are illustrated and discussed as a series of steps for purposes of explanation, the present disclosure is not limited to the particular illustrated order or to performance of every illustrated step in every implementation. One or more steps can be omitted, repeated, combined, subdivided, or performed in a different order, so long as the relevant data acquisition, association, metric determination, and/or recommendation functions are achieved. For example, certain preprocessing, event-detection, phase-identification, bilateral-comparison, baseline-generation, representative-data-set generation, and recommendation-related operations can be performed iteratively, in parallel, on a per-step basis, on a per-segment basis, or after completion of an activity. Similarly, certain determinations described as being performed by a computing device or remote server can additionally or alternatively be performed in whole or in part by one or more monitoring devices 18.

In some implementations, analytics can be distributed across the first monitoring device 20, the second monitoring device 22, the host computing device 62, and the remote server 64. For example, the first monitoring device 20 and the second monitoring device 22 can generate stride data locally. The host computing device 62 can pair first-foot and second-foot records and perform selected synchronization, display, and coaching functions. The remote server 64 can perform historical aggregation, representative-data-set generation, score normalization, anomaly detection, recommendation ranking, and training-readiness analytics. In other implementations, one or more such operations can be reallocated according to latency, available power, communication availability, memory usage, user settings, or privacy constraints.

### Stride Detection Algorithm

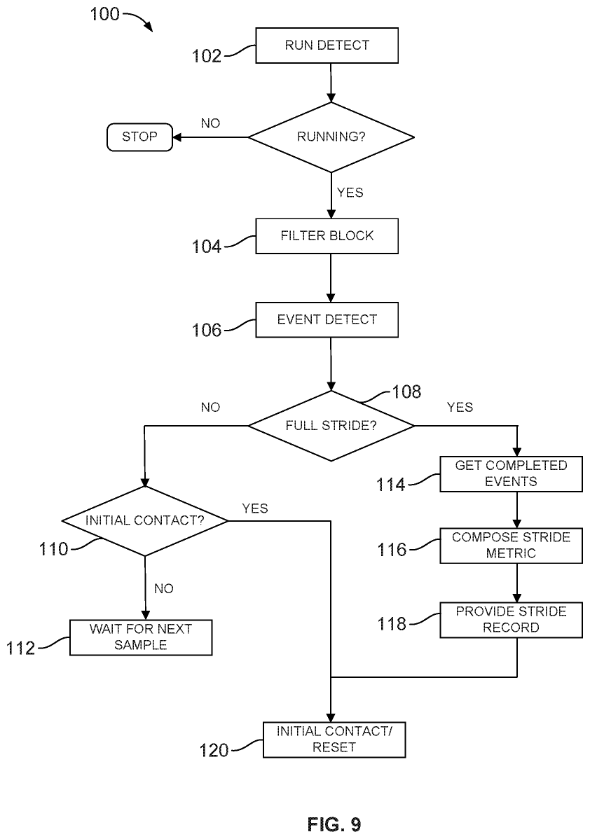

Referring to FIG. 9, a process 100 is illustrated for on-device stride detection and stride record generation. Process 100 can be performed by the processor 24 of the first monitoring device 20 and/or the second monitoring device 22 in real time during an activity, operating on inertial data acquired from the respective IMU 28 at a sample rate of approximately 480 Hz, for example. In some implementations, two parallel data streams can be prepared from the raw IMU data: a sample stream used for run detection, and a sample stream used for the run processing pipeline including filtering, event detection, and stride metric computation.

At 102, a run detection determination is made. The processor 24 can evaluate whether running activity is currently detected based on output from the machine-learning core or hardware decision tree classifier of the IMU 28 as described above with respect to the power mode and wake logic. In some implementations, run detection can be performed using a pedometer cadence value. In other implementations, run detection can use a custom algorithm also based on cadence and incorporating additional factors evaluated by the machine-learning core of the IMU 28. The machine-learning core can take raw sensor signals, apply pre-filtering, evaluate certain conditions of those signals using a decision tree that has been trained offline and pre-loaded onto the IMU 28, and classify activity types and movement qualities, outputting a classification result and generating an interrupt when the activity state changes, such as transitioning from stationary to walking, from walking to running, or from running back to a non-running state. If running is not detected, the processor 24 can stop stride processing and return to a non-recording state. If running is detected, the process 100 continues to step 104.

At 104, a filter block is applied to the raw inertial data received from the IMU 28. The filter block can process the raw inertial data through a sequence of transformations that estimate stance phase and orientation.

At 106, event detection is performed. The processor 24 can analyze the filtered inertial data to identify gait events comprising initial contact (IC), midstance (MS), and toe-off (TO). Initial contact can be identified from characteristic acceleration and angular velocity signatures corresponding to the onset of foot-ground contact. Toe-off can be identified from the inertial signature corresponding to the end of ground contact and the onset of the swing phase.

At 108, a determination is made as to whether a full stride is ready. A full stride can be considered ready when a complete sequence comprising an initial contact event, a midstance event, a toe-off event, and a subsequent initial contact event has been identified for the current stride-represented locomotion interval. A stride can be defined as the period from one initial contact event to the next initial contact event of the same foot. If a full stride is not yet ready, the process 100 evaluates at 110 whether an initial contact event has been detected for the current processing cycle. If an initial contact event has been detected, the process 100 proceeds to step 120 discussed below. If no initial contact event has been detected, the process 100 proceeds to step 112, where the processor 24 waits for the next inertial data sample before returning to step 106.

At 114, when a full stride is determined to be ready, the completed events for the current stride are retrieved. The completed events can include the timestamps and associated inertial data features for the initial contact event, midstance event, and toe-off event of the completed stride.

At 116, stride metrics are composed from the completed events and the filtered inertial data for the current stride. The stride metrics can include one or more of timestamps for initial contact, midstance, and toe-off; pronation excursion; foot strike angle; stride length; stride time; accumulated distance; accumulated velocity; underlying acceleration and angular velocity values; values derived therefrom such as Euler angles; and side information. In some implementations, the stride metrics can be stored using the following fields: initial contact timestamp; midstance timestamp; toe-off timestamp; pronation excursion in degrees, representing the angular rotation of the foot about its longitudinal axis between initial contact and midstance; foot strike angle in degrees, representing the angle of the foot relative to the ground at initial contact; stride distance in meters, representing a running sum reset at the end of each stride; stride velocity in centimeters per second; and a left/right side indicator. The pronation excursion and foot strike angle values can be determined on-device because they depend on raw inertial signals that are not retained after stride compression and cannot be reconstructed from the stored stride record alone. The stride metrics can be composed on a per-stride basis from inertial data acquired during the stance phase and swing phase of the completed stride. At least some raw and intermediate per-sample processing values can be discarded after the stride metrics have been composed, improving memory efficiency relative to continuous raw data storage.

At 118, the stride record is emitted. The composed stride metrics can be written to non-volatile memory 30 as a stride data record for the completed stride, stored as a millisecond offset from a run-start timestamp. Stride data records can be stored sequentially from oldest to newest within a workout, with each workout separated by a header. Each segment of a workout can be preceded by a segment header. In some implementations, a new segment header can be created when running resumes following a pause of more than approximately one minute but less than approximately ten minutes, such that the data from before and after the pause are stored within the same workout record but as distinct segments. A pause of more than approximately ten minutes can cause the current workout to be closed and any subsequent running to be stored as a new workout record, as described above. Recording can cease if the available memory 30 reaches capacity until the user connects the monitoring device 18 to the host computing device 62 and data is downloaded. Device logs, which can capture system state information including noteworthy events, warnings, and errors at the time of occurrence, can be configured to store specific event types and can be permitted to overwrite older log entries when log storage reaches capacity. The emitted stride record can also be made available for optional real-time streaming to the host computing device 62 via the wireless communication architecture 32 if a connection is active, at an effective data rate corresponding to stride frequency rather than raw IMU 28 sample rate.

At 120, following emission of the stride record, a notification of initial contact is issued, stride distance accumulators are reset for the next stride, and the stance gate is updated to prepare event detection logic for the subsequent stride cycle. The process 100 then returns to step 102 to evaluate continued running detection for the next stride.

In some implementations, the embedded state machine of the IMU 28 can additionally be used for gesture detection to initiate device bonding or other security-related operations. After receipt of certain commands or after connection attempts from the host computing device 62, the monitoring device 18 can trigger a gesture detection routine in which the monitoring device 18 must be held in a specific orientation, such as inverted and stationary, for a threshold period such as approximately two minutes, before the IMU 28 indicates that the gesture has been detected and the monitoring device 18 proceeds with the requested operation.

### On-Device Stride Data Determinations

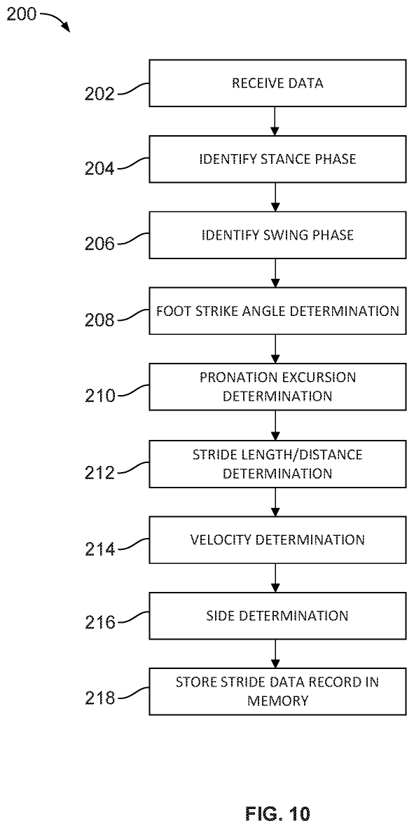

Referring to FIG. 10, a process 200 is illustrated for on-device stride data determinations performed by the processor 24 of the monitoring device 18. Process 200 can be performed in conjunction with process 100 and can describe in further detail the computations performed to derive stride metrics from the filtered inertial data and completed gait events identified during stride detection.

At 202, filtered inertial data and completed gait events from process 100 are received by the stride metric computation logic of the processor 24. The filtered inertial data can include orientation-corrected acceleration and angular velocity data for the completed stride.

At 204, a stance phase is defined between the identified initial contact event and the identified toe-off event for the current stride. Inertial data samples falling within the stance phase interval can be tagged as stance-phase data. The duration of the stance phase can be computed as a stance time value and stored as a per-stride metric. The initial contact, midstance, and toe-off times can be stored. In some implementations, the stance phase can also be used to update the drift correction state and orientation estimate for the current stride.

At 206, a swing phase is defined between the identified toe-off event and the subsequent initial contact event. Inertial data samples falling within the swing phase interval can be tagged as swing-phase data. The duration of the swing phase can be computed as a swing time value and stored as a per-stride metric. A flight time value, corresponding to the interval during which neither foot is in contact with the ground, can additionally be estimated from the swing phase data where bilateral timing information is available.

At 208, foot strike angle is determined from inertial data acquired at or around the initial contact event. The foot strike angle can be computed from the orientation of the monitoring device 18 at the moment of initial contact.

At 210, pronation excursion is determined from inertial data acquired during the stance phase. Pronation excursion can be stored as a per-stride metric. Pronation excursion and foot strike angle are determined on-device because those values can depend on raw inertial signals that are not retained after stride compression.

At 212, stride length and accumulated distance are determined from inertial data acquired during the stride. Accumulated distance can be updated by summing stride length values across successive strides. Both stride length and accumulated distance can be stored as per-stride metrics.

At 214, accumulated velocity is determined from inertial data acquired during the stride. Stride velocity can be estimated from the integrated acceleration over the stride interval after drift correction, and accumulated velocity can be updated accordingly. Stride velocity can be stored as a per-stride metric and can be used in downstream bilateral data fusion.

At 216, side information is determined. The side classification can be stored with the stride data record to assign an overall left-foot or right-foot identity to the monitoring device 18 for the current activity.

At 218, the composed stride metrics including stance time, swing time, foot strike angle, pronation excursion, stride length, accumulated distance, accumulated velocity, timestamps for initial contact, midstance, and toe-off, underlying acceleration and angular velocity values, Euler angles, and side information can be packaged as a stride data record and passed to step 118 of process 100 for storing in memory 30 and optional real-time streaming to the host computing device 62.

In some implementations, the host computing device 62 can calculate additional metrics from the stride data records transmitted by the first monitoring device 20 and the second monitoring device 22, and can match the left and right stride data streams for bilateral analysis. The host computing device 62 can provide the combined data to the remote server 64, which can derive an expanded set of metrics from the uploaded stride data. Metrics derived at the host computing device 62 or remote server 64 from the uploaded stride data can include one or more of stride duration, swing duration, flight duration, stride frequency, cadence, stance duration, step duration, step frequency, duty factor, spring ratio, estimated peak vertical ground reaction force, weighted impulse, stride validity index, and pace zone classification. Cadence can be expressed as steps per minute and can be derived from the step frequency of the uploaded stride records. In some implementations, a stride validity index can be determined to represent the trustworthiness or data quality of each stride record. In some implementations, the stride validity index can be based on one or more signal quality indicators, outlier flags, or filter state variables associated with the stride record. A weighted impulse can be determined to represent the mechanical stress experienced by the body during that stride, as described further below. These derived values can supplement the on-device stride metrics to form a complete per-stride data record for downstream analytics.

### Bilateral Association and Stride Event Identification

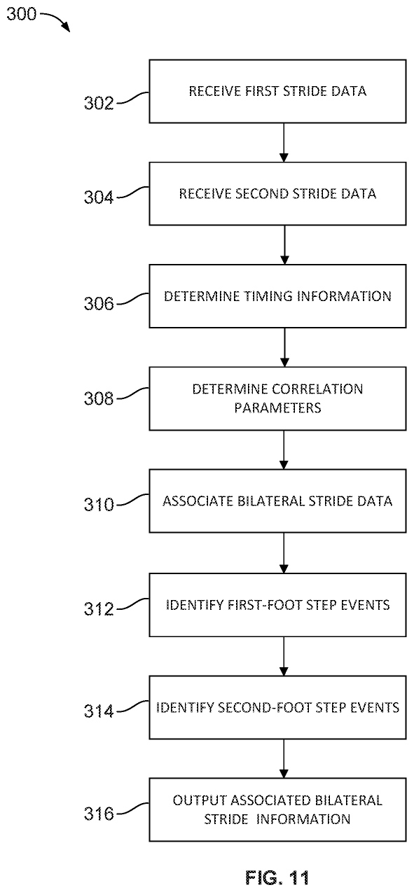

Referring to FIG. 11, a process 300 is illustrated for associating first stride data and second stride data and identifying bilateral stride event information. Process 300 can be performed by the host computing device 62, the remote server 64, or a combination thereof after upload and synchronization of stride data records from the first monitoring device 20 and the second monitoring device 22.

At 302, first stride data is received from the first monitoring device 20. The first stride data can include stride data records comprising timestamps, locally derived metrics, and side information as described above with respect to processes 100 and 200. The first stride data can be stored locally at the first monitoring device 20 and later transferred to the host computing device 62 in one or more records or segments. The received first stride data can then be staged for bilateral association and downstream gait analysis.

At 304, second stride data is received from the second monitoring device 22. The second stride data can include stride data records comprising timestamps, locally derived metrics, and side information corresponding to the second foot. The second stride data can be uploaded together with data indicating run identity, segment identity, or local timing information. The second stride data can then be prepared for association with the first stride data.

At 306, timing information associated with the first stride data and the second stride data is received or determined. The timing information can include timestamps, values from real-time clock 38, step-event timing, upload-time alignment data, host-clock information, drift information, or combinations thereof. The host computing device 62 or the remote server 64 can normalize or reformat the timing information into a common frame of reference using the system clock of the host computing device 62 together with timing information received from the first monitoring device 20 and the second monitoring device 22. The timing information can provide the basis for determining correlation parameters that associate the first stride data with the second stride data according to relative timing.

At 308, correlation parameters are determined based on relative timing between the first stride data and the second stride data. The host computing device 62 can determine the correlation parameters using timestamps, detected step-event timing, clock-offset information, drift information, phase information, or combinations thereof. The correlation parameters can include an offset value, a phase-correction value, a drift-correction value, one or more synchronization markers, one or more event timing relationships, or combinations thereof. In some implementations, a coarse alignment can be determined at the host computing device 62 based on run-start timing or real-time clock 38-derived timing information, while additional refinement of offset correction, drift correction, phase correction, or other bilateral alignment can be performed at the remote server 64 after upload. In some implementations, different correlation parameters can be determined for different segments of an activity, for example where drift, missing data, staggered wake-up behavior, or changing timing confidence suggests that segmented alignment is preferable to a single activity-wide alignment. Step events and contact events can be used as alignment anchors, particularly where the respective monitoring devices 20, 22 began recording at different times or where timing confidence varies across an activity, and first-foot and second-foot time streams can be interpolated onto a common time grid using linear interpolation to create a fused bilateral data set.

At 310, the first stride data is associated with the second stride data according to the correlation parameters. Associating the data can include time-aligning the first stride data and the second stride data into a shared time base. The result of step 310 is an associated bilateral data set in which the first stride data and the second stride data are correlated according to relative timing and are suitable for bilateral gait analysis.

At 312, first-foot step events are identified from the first stride data. The first-foot step events can be identified from the timestamps and event markers contained within the stride data records, including initial contact, midstance, and toe-off events. The identified first-foot step events can then be correlated with the associated bilateral data set.

At 314, second-foot step events are identified from the second stride data using methods consistent with those used for the first-foot step events, such that bilateral comparison is performed on a consistent basis. The identified second-foot step events can then be paired temporally with the first-foot step events.

At 316, associated bilateral stride event information is output for downstream analysis. The output can include the first stride data, the second stride data, the correlation parameters, the first-foot step events, and the second-foot step events in a common analytical structure. The output can be provided to a phase-analysis routine, a bilateral-symmetry routine, a baseline-generation routine, or combinations thereof. The output of process 300 thereby forms a foundational bilateral dataset for subsequent analytics.

### Phase-Based Gait Metric Determination

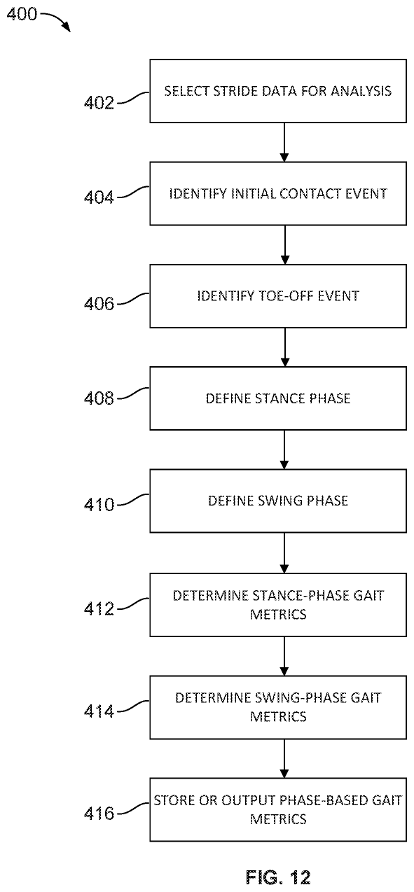

Referring to FIG. 12, a process 400 is illustrated for phase-based gait metric determination. Process 400 can use the associated bilateral data set from process 300 and can be performed after process 300, although in other embodiments portions of process 400 can be performed in parallel with step-event detection or local device processing.

At 402, a stride data record or stride-represented locomotion interval of the first foot or second foot is selected for analysis. The selected stride data record or stride-represented locomotion interval can be defined using one or more identified step-event boundaries from process 300. Stride-record or stride-represented interval selection can occur for each detected stride in sequence, for a subset of steps, or for steps within a selected time window or activity segment. In some implementations, a stride record is treated as complete after initial contact, midstance, toe-off, and a subsequent initial contact have been identified. The selected stride data record or stride-represented locomotion interval is then used as a basis for phase-specific event and metric determination.

At 404, an initial contact event is identified. The initial contact event can correspond to an onset of ground contact by the foot and can be identified from the initial contact timestamp and associated inertial features contained within the stride data record, including acceleration features, angular-velocity features, zero-crossings, threshold crossings, pattern matching, machine-learning classification, or combinations thereof. Multiple candidate initial-contact events can be evaluated and filtered according to confidence or consistency rules. The resulting initial contact event can define a leading boundary of a stance phase.

At 406, a toe-off event is identified. The toe-off event can correspond to an end of ground contact by the foot and can be identified from the toe-off timestamp and inertial signatures contained within the stride data record. The toe-off event can be selected from among multiple candidates using temporal constraints, expected phase durations, or inter-event consistency criteria. The toe-off event can define a transition from stance to swing phase. A midstance event can additionally be identified between the initial contact event and the toe-off event and can correspond to a stationary or reduced-motion period.

At 408, a stance phase is defined between the initial contact event and the toe-off event. The stance phase can be treated as a period during which the corresponding foot is in contact with the ground or otherwise bearing load. Inertial data samples and stride metric values falling between the initial contact event and the toe-off event can be tagged as stance-phase data for the selected stride-represented locomotion interval. That stance-phase data can then be used to determine one or more stance-phase gait metrics.

At 410, a swing phase is defined between the toe-off event and a subsequent initial contact event. The swing phase can be treated as a period during which the corresponding foot is off the ground and advancing toward a subsequent contact. Inertial data samples and stride metric values falling between the toe-off event and the subsequent initial contact event can be tagged as swing-phase data for the selected stride-represented locomotion interval. That swing-phase data can then be used to determine one or more swing-phase gait metrics.

At 412, one or more stance-phase gait metrics are determined from stride data acquired during the stance phase. The stance-phase gait metrics can include one or more of stance time, foot strike angle, pronation excursion, contact time, or a ground-reaction-force-related value. The determined stance-phase gait metrics can be stored as per-stride-derived values and/or aggregated over multiple stride data records for later use in bilateral comparison and baseline generation. In some implementations, an estimated peak vertical ground reaction force can be determined for each stride. The ground reaction force can be modeled as a function of time t during the stance phase.

At 414, one or more swing-phase gait metrics are determined from stride data acquired during the swing phase. The swing-phase gait metrics can include one or more of flight time, stride length, cadence contribution, vertical oscillation, or other motion-related values. The determined swing-phase gait metrics can likewise be stored as per-stride-derived values and/or aggregated over multiple stride data records for subsequent bilateral comparison and recommendation generation.

At 416, the determined phase-based gait metrics are stored or output. The output can include a mapping between the selected stride data record or stride-represented locomotion interval, its stance-phase gait metrics, and its swing-phase gait metrics. The output can be provided to a bilateral-comparison routine, a baseline-profile routine, a user-interface generation routine, or combinations thereof. Process 400 thereby provides the associated bilateral data set with explicit phase-based gait metrics that can be reused by other analytics. In some implementations, the stored or output phase-based gait metrics can include per-stride-derived values, summary values for a selected segment, split-based values, rolling values, or combinations thereof. In some implementations, additional metrics can be derived from the phase-based gait metrics, including stride duration, swing duration, flight duration, stride frequency, cadence, step duration, step frequency, duty factor, spring ratio, and further derived values. In some implementations, a duty factor can be computed as a ratio of stance duration to stride duration, and a spring ratio can be computed as a ratio of flight duration to stride duration or a related biomechanical measure of elastic energy storage and return.

### Bilateral Symmetry and Gait Characteristic Determination

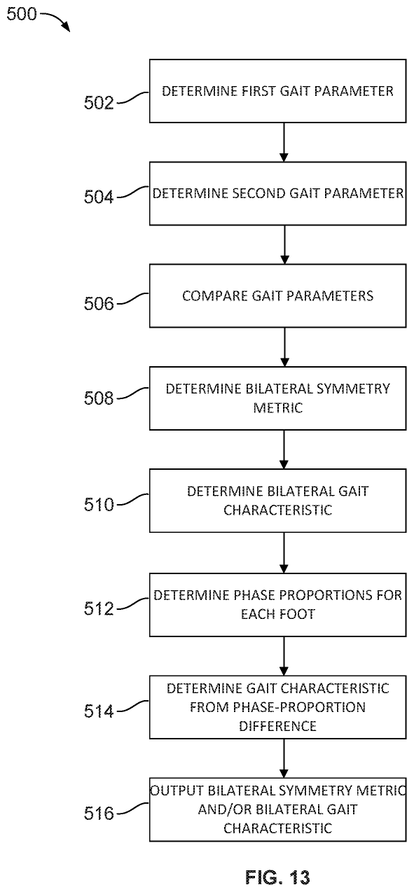

Referring to FIG. 13, a process 500 is illustrated for bilateral symmetry and gait-characteristic determination. Process 500 can use the associated bilateral data set from process 300 and one or more phase-based gait metrics from process 400.

At 502, a first gait parameter is determined for the first foot. The first gait parameter can include stance time, swing-phase duration, stride length, pronation excursion, impact-related value, or another per-stride-derived or aggregated gait metric derived from the first stride data. The first gait parameter can be determined for an individual stride data record or stride-derived locomotion interval, for a plurality of stride data records, or for a selected segment of activity. The first gait parameter provides a first-foot basis for bilateral comparison.

At 504, a second gait parameter is determined for the second foot. The second gait parameter can correspond to the same type of gait parameter as the first gait parameter, such that the two values can be compared meaningfully. The second gait parameter can be determined for an individual stride data record or stride-derived locomotion interval, for a plurality of stride data records, or for a selected segment of activity using the second stride data. The second gait parameter provides a second-foot basis for bilateral comparison.

At 506, the first gait parameter and the second gait parameter are compared. The comparison can include a difference, ratio, normalized difference, asymmetry index, temporal offset, or another bilateral comparison operation. Comparisons can be performed step-by-step, over aligned sequences of steps, or over aggregated windows of activity. The resulting comparison value characterizes a bilateral relationship between the two feet. In some implementations, steps 502 and 504 can be performed in parallel, and the comparison of step 506 can be updated iteratively as additional first-foot and second-foot data becomes available.

At 508, a bilateral symmetry metric is determined based on the comparison. The bilateral symmetry metric can quantify a difference in a physical gait parameter between the first foot and the second foot. Separate bilateral symmetry metrics can be determined for stance time, swing-phase duration, impact-related values, stride length, or other gait metrics. The bilateral symmetry metric can then be used for deviation detection, baseline comparison, recommendation generation, or combinations thereof. The bilateral symmetry metric can be expressed as a raw difference, normalized difference, ratio, asymmetry score, percentage difference, or other value representing left-right disparity of the relevant gait parameter.

At 510, a bilateral gait characteristic is determined based on a temporal relationship between the first-foot step events and the second-foot step events. The bilateral gait characteristic can reflect timing alternation, coordination, phase relationship, or another cross-foot movement characteristic. The bilateral gait characteristic can be derived using the bilateral symmetry metric, the event timing information, or combinations thereof. The bilateral gait characteristic can provide a broader analytical summary than any single bilateral symmetry metric alone. In some implementations, the bilateral gait characteristic can reflect an interaction among multiple bilateral symmetry metrics and/or multiple temporal relationships rather than being limited to a single compared value.

At 512, for a plurality of stride-represented locomotion intervals, a proportion of each stride-represented locomotion interval corresponding to the stance phase and/or the swing phase is determined for each foot. The proportion can be calculated as a duration of the phase divided by an overall locomotion-interval duration. The proportion can be determined for individual stride-represented locomotion intervals and then averaged or otherwise summarized across a set of stride data records. Those proportions can reveal left-right differences in how the gait cycle is apportioned between stance and swing.

At 514, a gait characteristic is determined based on a difference between the proportions for the first foot and the second foot. The difference can be used to quantify stance-phase imbalance, swing-phase imbalance, or both. The gait characteristic determined at 514 can supplement or refine the bilateral symmetry metric and/or the bilateral gait characteristic determined earlier in process 500. The resulting characteristic can be retained as an explicit bilateral feature for downstream use. In some implementations, bilateral symmetry metrics and pace-conditioned gait features form inputs to an anomaly-detection routine that determines whether a current movement pattern differs from a user-specific baseline for a corresponding pace range.

At 516, the bilateral symmetry metric and/or the bilateral gait characteristic is output. The output can be provided to a baseline-generation routine, a recommendation-generation routine, a user-interface routine, or combinations thereof. Process 500 thereby generates bilateral symmetry metrics and bilateral gait characteristics suitable for individualized monitoring and coaching. In some implementations, a form assessment or running signature can be generated from the bilateral symmetry metrics and gait characteristics to indicate how the user runs, how that pattern affects the body, and to recommend accommodations such as strength training or technique modifications. The form assessment can include symmetry metrics, pronation characteristics, and historical trends to show performance over time. Recommendations relative to a demographic of other runners can additionally be generated.

### Baseline Gait Profile, Cumulative Impact, and Recommendation Generation

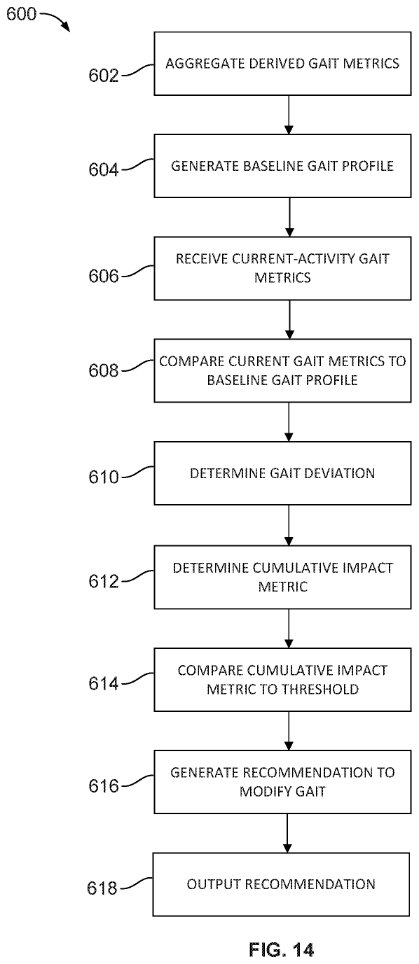

Referring to FIG. 14, a process 600 is illustrated for baseline-gait-profile generation, cumulative-impact determination, and recommendation generation. Process 600 can operate on historical and current analytical outputs from one or more prior processes.

At 602, derived gait metrics are aggregated across a plurality of activities of the user. The aggregated gait metrics can include one or more bilateral symmetry metrics, bilateral gait characteristics, phase-based gait metrics, impact-related values, or other derived parameters. The metrics can be grouped by activity type, training phase, footwear condition, or other contextual information. The aggregated metrics provide a historical dataset from which a representative user profile can be generated.

At 604, a baseline gait profile is generated based on the aggregated derived gait metrics. The baseline gait profile can include average values, ranges, distributions, variability measures, confidence measures, or modeled relationships among multiple gait parameters. The baseline gait profile can be user-specific and can be updated over time as more activity data becomes available. The baseline gait profile serves as a reference against which current-activity behavior can be assessed. In some implementations, the baseline gait profile can be updated incrementally over time, can exclude outlier activities, can be conditioned on activity type, footwear type, terrain, fatigue state, or user history, and can include one or more individualized expected ranges for bilateral symmetry metrics, phase-based gait metrics, impact-related values, or combinations thereof. In some implementations, separate baseline gait profiles are maintained for different pace ranges or speed ranges so that current gait metrics are compared against pace-conditioned baseline values rather than a single global baseline. In some implementations, pace zones can be established based on a pace or race time provided at initial device setup, from which easy, medium, and hard pace zones can be determined, with each stride classified into a pace zone for aggregation and baseline modeling purposes.

At 606, derived gait metrics from a current activity are received. The current-activity gait metrics can include bilateral symmetry metrics, bilateral gait characteristics, phase-based gait metrics, impact-related values, stride-related values, or other outputs from prior processes. The received gait metrics can correspond to the activity as a whole, selected segments of the activity, or per-step values. Those current gait metrics provide a current snapshot of the user's movement behavior.

At 608, the derived gait metrics from the current activity are compared to the baseline gait profile. The comparison can include direct differences, normalized differences, trend analysis, statistical comparisons, confidence-based assessments, or combinations thereof. Multiple current gait metrics can be compared simultaneously to multiple baseline parameters. The comparison reveals whether the user's current gait behavior aligns with or deviates from the user's typical gait behavior as represented by the baseline gait profile. In some implementations, the comparison is performed using a current pace range and a corresponding baseline gait profile for that pace range.

At 610, a determination is made as to whether one or more gait characteristics deviate from a typical gait pattern of the user. This determination can be based on one or more thresholds, statistical criteria, confidence criteria, or learned decision rules. Different deviation criteria can apply to different gait metrics, activity contexts, or user conditions. The deviation determination can be used to gate whether a recommendation to modify gait will be generated. A deviation can be determined based on a departure from one or more individualized baseline ranges, a trend over time, a change from a prior segment of the same activity, a threshold comparison, or combinations thereof. In some implementations, the deviation determination forms part of a form-radar or anomaly-detection analysis that identifies departures in one or more of cadence, flight time, ground-contact time, symmetry, pronation-related values, foot strike angle, or combinations thereof. In some implementations, the form radar analysis can detect deviations both after the fact and in real time, and can generate recommendations relative to a demographic of comparable runners in addition to the user's own baseline.

At 612, a cumulative impact metric is determined based on a plurality of stride-derived impact-related values. The cumulative impact metric can be determined by summing, integrating, weighting, or otherwise aggregating per-stride or stride-derived impact-related values across part or all of the current activity. In some implementations, a per-stride weighted impulse (WI) can be computed. A cumulative weighted impulse (CWI) for a given run can be obtained by summing the individual weighted impulse values across all strides of the run, according to:

$$CWI = {\sum }_{i = 1}^{n} {WI}_{i}$$

where n is the total number of strides, representing the total mechanical load of the run. The cumulative impact metric can reflect mechanical stress accumulated during the activity and can then be evaluated relative to prior activity history or a user-specific limit.

At 614, the cumulative impact metric is compared to a threshold based on prior activities of the user. The threshold can be fixed, adaptive, activity-specific, user-specific, population-informed, or otherwise derived from historical data. The comparison can reveal whether the current activity is imposing more or less mechanical stress than expected for the user. The threshold comparison can independently support recommendation generation or can be combined with the baseline comparison of step 608. In some implementations, the cumulative impact metric is normalized based on historical activity data of the user to produce an impact score representing mechanical load for the activity. In some implementations, the impact score can be used to prescribe recommended run length or pace, to identify gait issues in real time and communicate changes to avoid injury, and to build a model of the relationship between running speed and ground reaction force based on historical data, enabling prediction of expected impact scores for planned activities even when the user is not wearing the monitoring device 18.

At 616, a recommendation is generated based on the baseline gait profile, the cumulative impact metric, the bilateral symmetry metric, or combinations thereof. The recommendation can be a recommendation to modify the gait of the user and can include guidance to reduce impact, improve symmetry, alter pacing, modify stride length, adjust cadence, take a recovery action, or otherwise respond to the detected condition. The recommendation can be prioritized, ranked, filtered, or personalized before output. In some implementations, the recommendation can be a personalized daily recommendation based on the user's training objective. In some implementations, the comparison of step 608, the deviation determination of step 610, and the recommendation generation of step 616 can be performed repeatedly during an ongoing activity as additional gait metrics are received. In other implementations, one or more of those steps can be deferred until after completion of the activity. Further, generation of the recommendation can occur even if one of the illustrated analytical inputs is unavailable, such as where the recommendation is based on the cumulative impact metric without use of a current bilateral symmetry metric, or vice versa. In some implementations, the recommendation can be selected from among a plurality of candidate recommendations based on severity of deviation, confidence of the underlying determination, cumulative impact, user preferences, prior recommendation history, or combinations thereof. In some implementations, the recommendation includes an instruction to modify cadence, speed, impact, or bilateral symmetry during an ongoing activity. In some implementations, current real-time coaching uses cadence or step frequency, such as through a metronome output, while other implementations additionally or alternatively coach toward reduced impact or improved efficiency.

At 618, the recommendation is output during the activity and/or after the activity. The recommendation can be output in real time through an audio output, a visual output, a haptic output, or a combination thereof. The recommendation can additionally or alternatively be stored for later review, summarized in a report, or integrated into a training plan. Process 600 thereby converts analytics into user-facing guidance, including a real-time recommendation to modify gait in some implementations. In some implementations, the recommendation can be suppressed, delayed, combined with another recommendation, or reformulated into a different presentation mode based on context of the activity, user settings, confidence level, or repetition-control logic.

In some implementations, historical stride data is additionally compressed into a representative data set by grouping strides into pace ranges or speed ranges and retaining a subset of stride records for each range. In some implementations, a predetermined number of strides per pace zone can be retained as representative of the movement pattern at that pace, with pace zones determined from the user's provided race time or pace at initial device setup and each stride categorized as easy, medium, or hard accordingly. The representative data set can preserve modeled relationships, such as pace-versus-impact behavior, while reducing the number of historical stride records used for subsequent recommendation logic. By modeling the relationship between pace and ground reaction force from the representative data set, the system can generate more specific recommendations, such as suggesting a particular run distance based on the user's pace and expected impact at that pace, and can estimate impact scores for planned activities. In some implementations, the representative data set is used to determine an expected impact metric for a proposed activity, such as a planned run distance at a selected pace, and to generate a training prescription responsive to user goals and expected mechanical load.

In some implementations, a readiness score or a resiliency score is determined from a plurality of historical impact scores corresponding to prior activities of the user. In some implementations, the resiliency score can be based on models to generate a score indicating the user's readiness for training, updated on a daily basis. The readiness score or resiliency score can be based on one or more contributors comprising: acute load covering approximately the last seven days of training, evaluating the trend and identifying large spikes in impact relative to recent history; chronic load covering approximately the last three weeks of training, evaluating the overall training trend to identify declining performance despite possible short-term spikes; training variability modeled over approximately thirty days to identify outliers in day-to-day variability relative to the user's typical pattern; and overall load reflecting a cumulative fitness score relative to a threshold above which tissue breakdown risk increases. The readiness score or resiliency score can be output to indicate whether the user is better suited for additional training, recovery, or rest.

In some implementations, an efficiency score can be determined to represent the efficiency of the user's movement through space. The efficiency score can be based on a relationship between mechanical work output and physiological effort. In some implementations, mechanical work output can be estimated from the stride data including ground reaction force estimates and stride length, and physiological effort can be represented by heart rate, such that the efficiency score reflects a ratio of estimated mechanical work in joules to heart rate, representing how hard the user is working to achieve a given movement result. Heart rate data for the efficiency score can be obtained from external sources such as a wearable heart rate monitor, a smart ring, or a smartwatch.

### Supplemental Inputs and User Interface

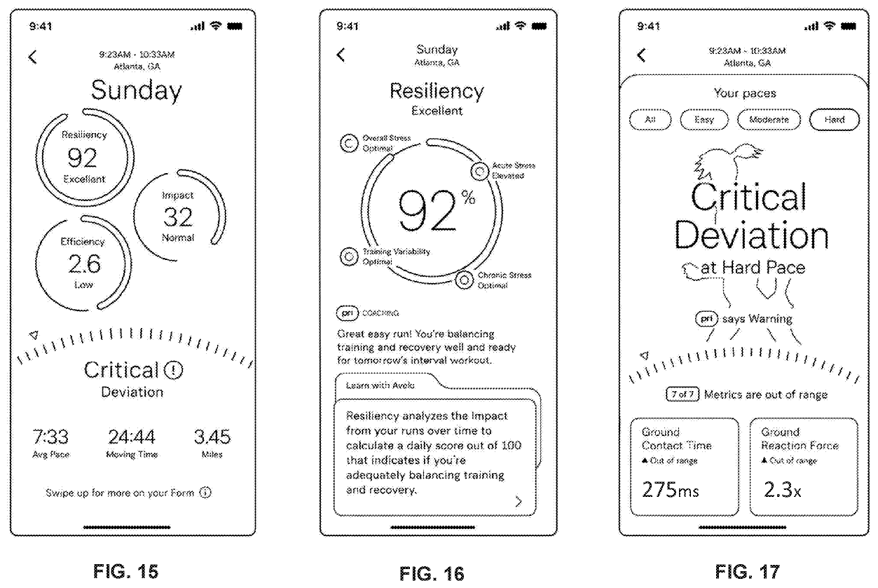

Referring to FIGS. 15-17, the host computing device 62 can present information through a user interface. FIG. 15 depicts an impact-related score interface, FIG. 16 depicts a resiliency or readiness interface, and FIG. 17 depicts a deviation, anomaly, or coaching interface. The user interface can present score information, resiliency information, deviation information, selected metrics, and recommendations based on the baseline gait profile, the cumulative impact metric, and the recommendation. The recommendation can be delivered via an audio output, a visual output, a haptic output, or a combination thereof through the user interface. The recommendation can be generated in real time during an ongoing activity of the user and delivered via at least one of an audio output or a visual display. In some implementations, the user interface can present current values, historical values, bilateral comparisons, trends over time, split-based summaries, baseline comparisons, alerts, recommendations, or combinations thereof.

Additional data sources can be used together with the first stride data and the second stride data to contextualize derived gait metrics or recommendation logic. Such additional data can include heart rate, blood oxygenation, weather, climate, altitude, pressure, sleep, VO2max, body temperature, hydration, lactic-threshold-related information, position information, self-reported user information, or combinations thereof. In some implementations, heart rate data can be obtained from external sources including wearable devices such as a smart ring, smartwatch, or chest-strap heart rate monitor, and can be used together with stride data to determine the efficiency score and to improve readiness and training prescription analyses. In some implementations, one or more such additional data sources can be used to modify a threshold, select a recommendation, interpret a detected deviation, or otherwise contextualize bilateral gait analysis. Additional data can be obtained from integrated third-party platforms, health-data repositories, fitness devices, or user-entered data sources.

The monitoring devices 18 and/or external computing systems can determine impact-force and stride-length related values from the associated data sets. A foot-impact profile can track a known or expected curvature, and an underlying impact-force value derived from acceleration measurements can be further processed to estimate a ground-reaction-force related value. In some implementations, the estimated peak vertical ground reaction force for each stride can be determined, providing a per-stride force estimate from which a weighted impulse and cumulative impact score can be derived. From inertial data acquired during the stance phase, an impact-force value can be determined for each of a plurality of stride-represented locomotion intervals. From inertial data acquired during the swing phase, a stride length can be determined for each of the plurality of stride-represented locomotion intervals. The impact-force value and the stride length corresponding to one or more steps can be used together to determine the bilateral gait characteristic and/or another gait characteristic of the user. In some implementations, impact-related values can be determined for individual stride-represented locomotion intervals and then aggregated over a plurality of stride-represented locomotion intervals, while stride-length-related values can be determined for corresponding stride-represented locomotion intervals or segments and analyzed together with the impact-related values to identify movement patterns, efficiency characteristics, asymmetries, fatigue-related changes, or recommendation triggers.

In some implementations, heart-rate-related inputs can improve efficiency analysis, readiness analysis, or training-prescription generation; weather- and terrain-related inputs can improve interpretation of pace-conditioned gait deviations; sleep- and recovery-related inputs can improve resiliency determinations; and body-mass-related inputs can improve force estimation and impact normalization. In some implementations, impact-force-related values can be determined using one or more acceleration-derived features, inertial signals, phase durations, regression-based estimates, user-specific features, or combinations thereof.

### Additional Applications

Although the present disclosure has been described in the context of a monitoring device 18 associated with a footwear article 12 and positioned within or relative to the footwear article 12, the methods, systems, and devices of the present disclosure can be applied to a variety of applications, such as running, as well as other forms of exercise, including walking and active sports, and therapeutic applications such as inpatient or outpatient monitoring. The improved inertial measurements enabled by the present disclosure can be utilized for other metrics, such as impacts that occur during sports such as tennis, golf, basketball, soccer, and the like, providing recommendations for pacing, efficiency, breaks, technique, and the like.

In exemplary therapeutic applications, a disposable footwear article insert or disposable footwear article can be provided to a patient, with relevant data and recommendations made available to the patient and treating physicians and staff. As another example, for active employment applications where an employee is often walking, standing, or moving, such as in building trades, warehousing, manufacturing, or service occupations, recommendations such as pace and breaks can be provided to the employee and/or employer. In any of these applications, the system can associate stride data from a first foot with stride data from a second foot according to relative timing, determine bilateral gait characteristics and phase-based gait metrics, and provide real-time and/or post-activity recommendations. In further implementations, the disclosed system can be used in injury-recovery programs, gait rehabilitation, elder-mobility monitoring, occupational ergonomics, sports-performance training, military or tactical training, and footwear-development or product-testing applications in which bilateral movement information and phase-based gait information are relevant. In some implementations, one or more of the historical-baseline, pace-conditioned anomaly, cumulative-impact, representative-data-set, readiness, resiliency, running-signature, or efficiency analyses described herein can be adapted to these non-running applications by using activity-appropriate event definitions, thresholds, and baseline groupings.

In some implementations, rehabilitation and injury-recovery use cases can emphasize asymmetry detection, pace-conditioned anomaly detection, and recommendation logic directed to safe return-to-activity progression. Occupational use cases can emphasize cumulative impact, fatigue-related changes, asymmetry accumulation over long work periods, and break recommendations. Product-development or footwear-testing use cases can emphasize cohort comparisons, pace-conditioned form profiles, impact distributions, and differences among footwear configurations. Team-sport or tactical use cases can emphasize acceleration, deceleration, cutting, asymmetry, and repeated-impact monitoring.

The foregoing description includes exemplary embodiments in accordance with the present disclosure. These examples are provided for purposes of illustration only, and not for purposes of limitation. It will be understood that the present disclosure may be implemented in forms different from those explicitly described and depicted herein and that various modifications, optimizations, and variations may be implemented by a person of ordinary skill in the present art, consistent with the following claims.

What is claimed is:

1. A system comprising:
a first self-contained monitoring device configured to be secured relative to a first foot of a user and to generate first stride data from inertial data acquired by a first inertial measurement unit during an activity;
a second self-contained monitoring device configured to be secured relative to a second foot of the user and to generate second stride data from inertial data acquired by a second inertial measurement unit during the activity; and
one or more computing devices separate from the first monitoring device and the second monitoring device, configured to:
receive the first stride data for the activity and the second stride data for the activity;
associate the first stride data with the second stride data based on relative timing between the first stride data and the second stride data during the activity to generate an associated bilateral data set;
determine, from the associated bilateral data set, a mechanical load metric representing cumulative physical stress experienced by the user during the activity; and
provide one or more representations of the mechanical load metric to the user.

2. The system of claim 1, wherein the one or more computing devices are further configured to determine, for each of a plurality of locomotion intervals represented in the associated bilateral data set, an impact-related value based on inertial data acquired during a stance phase of the respective locomotion interval, and wherein the mechanical load metric is based on an aggregation of the impact-related values across the plurality of locomotion intervals.

3. The system of claim 2, wherein the impact-related value for each locomotion interval is based on an estimated peak vertical ground reaction force.

4. The system of claim 1, wherein the one or more computing devices are further configured to normalize the mechanical load metric based on historical activity data of the user to produce an impact score, and to store the impact score in association with the activity for use in subsequent comparisons.

5. The system of claim 4, wherein the one or more computing devices are further configured to determine a resiliency score or readiness score from a plurality of historical impact scores corresponding to prior activities of the user, the resiliency score or readiness score reflecting at least two of acute training load, chronic training load, training variability, or overall accumulated load over respective historical windows.

6. The system of claim 1, wherein the one or more computing devices are further configured to generate, from historical stride data of the user, a representative data set by grouping stride records into pace zones and retaining a subset of stride records for each pace zone, and to use the representative data set to model a relationship between running pace and ground reaction force for the user and to determine an expected mechanical load for a proposed activity at a selected pace.

7. The system of claim 1, wherein the one or more computing devices are further configured to generate a recommendation for the user based at least in part on a comparison between the mechanical load metric and historical activity data of the user, wherein the one or more computing devices are further configured to establish a baseline gait profile for the user comprising, for each of a plurality of pace ranges, pace-conditioned expected ranges for one or more gait metrics, to compare one or more current gait metrics from the activity to the pace-conditioned expected ranges corresponding to a current pace range, and to generate the recommendation based at least in part on a determination of whether the current gait metrics satisfy a deviation criterion relative to the pace-conditioned expected ranges.

8. The system of claim 7, wherein the deviation criterion identifies departures in one or more of cadence, flight time, ground-contact time, bilateral symmetry, pronation excursion, or foot strike angle.

9. The system of claim 1, wherein associating the first stride data with the second stride data comprises performing a coarse alignment based on timing information from the first monitoring device and the second monitoring device, and performing a finer alignment using detected step events or contact events as alignment anchors.

10. The system of claim 1, wherein the first stride data and the second stride data each comprise at least timestamps for initial contact, midstance, and toe-off of a respective stride and at least pronation excursion and foot strike angle for the respective stride.

11. A system comprising:
a first monitoring device configured to be secured relative to a first foot of a user, to generate first stride data records from inertial data acquired by a first inertial measurement unit during an activity, and to store a first local data set comprising the first stride data records in a first local memory of the first monitoring device;
a second monitoring device configured to be secured relative to a second foot of the user, to generate second stride data records from inertial data acquired by a second inertial measurement unit during the activity, and to store a second local data set comprising the second stride data records in a second local memory of the second monitoring device; and
one or more computing devices configured to:
receive the first local data set from the first local memory and the second local data set from the second local memory;
associate the first local data set with the second local data set based on relative timing between the first local data set and the second local data set to generate an associated bilateral data set, the first local data set and the second local data set having been generated and stored independently by the first and second monitoring devices during the activity;
generate a baseline gait profile, the baseline gait profile comprising expected ranges for one or more gait metrics of the user;
determine, from the associated bilateral data set, a cumulative mechanical load metric for the activity;
compare one or more current gait metrics from the activity to the expected ranges of the baseline gait profile; and
generate a recommendation for the user based at least in part on the cumulative mechanical load metric and the comparison of the current gait metrics to the expected ranges.

12. The system of claim 11, wherein the cumulative mechanical load metric is determined by aggregating, across a plurality of locomotion intervals of the activity, impact-related values each based on an estimated peak vertical ground reaction force over a stance phase of the respective locomotion interval.

13. The system of claim 11, wherein the one or more computing devices are further configured to determine a resiliency score or readiness score from a plurality of historical impact scores corresponding to prior activities of the user, the resiliency score or readiness score reflecting at least two of acute training load, chronic training load, training variability, or overall accumulated load over respective historical windows.

14. The system of claim 11, wherein the one or more computing devices are further configured to generate, from historical stride data of the user, a representative data set by grouping stride records into pace zones and retaining a subset of stride records for each pace zone, and to use the representative data set to model a relationship between running pace and mechanical load for the user and to determine an expected mechanical load for a proposed activity.

15. The system of claim 11, wherein the baseline gait profile comprises, for each of a plurality of pace ranges, pace-conditioned expected ranges for one or more gait metrics, and wherein the one or more computing devices are configured to compare the current gait metrics to the pace-conditioned expected ranges corresponding to a current pace range of the activity.

16. The system of claim 11, wherein the first stride data records and the second stride data records each comprise at least timestamps for initial contact, midstance, and toe-off of the respective stride, and at least pronation excursion and foot strike angle of the respective stride, wherein pronation excursion and foot strike angle are determined on the respective monitoring device from raw inertial signals that are not retained in the first local data set or the second local data set after generation of the respective stride data record.

17. The system of claim 11, wherein the first local data set is retained in the first local memory of the first monitoring device and the second local data set is retained in the second local memory of the second monitoring device until the one or more computing devices have confirmed successful receipt and bilateral reconciliation of both the first local data set and the second local data set.

18. The system of claim 11, wherein the one or more computing devices are further configured to determine a bilateral symmetry metric quantifying a difference between one or more gait parameters of the first foot and corresponding gait parameters of the second foot, and wherein the recommendation is further based on the bilateral symmetry metric.

19. The system of claim 11, wherein the first monitoring device generates the first stride data records by real-time processing of the inertial data during the activity and stores the first local data set in the first local memory of the first monitoring device during the activity, and the second monitoring device generates the second stride data records by real-time processing of the inertial data during the activity and stores the second local data set in the second local memory of the second monitoring device during the activity.

20. A system comprising:
a first monitoring device configured to be secured relative to a first foot of a user, to acquire first inertial data from a first inertial measurement unit during an activity, and to generate and store in a first local memory first stride data records each comprising at least timestamps for initial contact, midstance, and toe-off of a respective stride of the first foot and at least pronation excursion and foot strike angle of the respective stride of the first foot determined from raw inertial signals that are not retained in the first local memory after generation of the respective first stride data record during the activity;
a second monitoring device configured to be secured relative to a second foot of the user, to acquire second inertial data from a second inertial measurement unit during the activity, and to generate and store in a second local memory second stride data records each comprising at least timestamps for initial contact, midstance, and toe-off of a respective stride of the second foot and at least pronation excursion and foot strike angle of the respective stride of the second foot determined from raw inertial signals that are not retained in the second local memory after generation of the respective second stride data record during the activity; and
one or more computing devices configured to:
receive the first stride data records from the first local memory and the second stride data records from the second local memory after the activity;
associate the first stride data records with the second stride data records based on relative timing, including performing a coarse alignment based on timing information derived from each of the first monitoring device and the second monitoring device and a finer alignment using detected step events or contact events as alignment anchors, to generate an associated bilateral data set;
determine, from the associated bilateral data set, an estimated peak vertical ground reaction force for each of a plurality of locomotion intervals;
determine, for each locomotion interval, an impact-related value based on the estimated peak vertical ground reaction force over a stance phase of the respective locomotion interval;
determine a cumulative impact metric for the activity by aggregating the impact-related values;
compare the cumulative impact metric to a threshold derived from historical activity data of the user;
establish a baseline gait profile comprising expected ranges for one or more gait metrics of the user, and compare one or more current gait metrics from the activity to the expected ranges; and
generate, based at least in part on the cumulative impact metric comparison and the gait comparison, a recommendation for the user.
